(19) United States
(12) Patent Application Publication
(10) Pub. No.: US 2024/0410905 A1
(43) Pub. Date: Dec. 12, 2024

(54) Method for survival prediction and for diagnosis and treatment of Type 2 Diabetes mellitus

(71) Applicant: Nordisk Hjertecenter ApS, Skodsborg (DK)

(72) Inventor: Søren Ballegaard, Skodsborg (DK)

(21) Appl. No.: 18/698,906

(22) PCT Filed: Oct. 6, 2022
(86) PCT No.: PCT/EP2022/077801

§ 371 Date: Apr. 5, 2024

(30) Foreign Application Priority Data
Oct. 6, 2021 (EP) 21201191.0
Apr. 26, 2022 (EP) 22169883.0

(51) Int. Cl. G01N 33/72

(52) U.S. Cl. CPC G01N 33/723; G01N 2800/042

(57) ABSTRACT

A novel method of predicting life expectancy in persons, in particular in persons that have an a priori reduced life expectancy, for instance if they suffer from ischemic heart disease (IHD). IHD-related depression, ANSD, type 2 diabetes mellitus is provided. Also disclosed is a related method for increasing life expectancy in human subjects, in particular in human subjects having an a priori reduced life expectancy. The disclosure also provides a method for evaluating the efficacy of drugs other treatment of ANSD. The present invention also provides a novel method for diagnosing and treating/preventing type 2 diabetes mellitus or IHD-related depression. Finally, the disclosure also provides a method for diagnosing autonomic homeostatic glucose dysregulation, and a novel method to re-establish normal homeostatic function, if disrupted, dysregulated or insufficient.

### FIELD OF THE INVENTION

The present invention relates to a novel method for prediction of death risk (expressed e.g. as a 5-year survival chance) in humans, in particular in human ischemic heart disease patients, as well as a method for diagnosis of type 2 diabetes mellitus. The invention further relates to a method of increasing survival rate, in particular in subjects having an a priori reduced survival risk and related to this also a method for maintaining humans at an increased survival rate and a method for treating type 2 diabetes mellitus. The invention also relates to methods for identification or evaluation of treatments ANSD and type 2 diabetes mellitus.

### BACKGROUND OF THE INVENTION

Autonomic nervous system dysfunction (ANSD) is known to be associated with multiple conditions, including stress, depression, insomnia, allergies, type 2 diabetes (T2D), and ischemic heart disease (IHD).

The autonomic nervous system (ANS) regulates functions of the human body by adjusting the balance between the sympathetic and parasympathetic nervous systems. The dynamic processes of adaptation in the face of adversity are orchestrated by a dynamic readjustment of the two systems known as resilience (McEwen, 2016; Carnevali et al., 2018). Autonomic nervous system dysfunction (ANSD) is defined as a reduction of this resilience, associated with morphological and functional changes of brain regions, e.g., hippocampus, amygdala, and prefrontal cortex (McEwen et al., 2016). ANSD may be defined as an imbalance in the ratio between parasympathetic and sympathetic nerve activity and appears as parasympathetic hypoactivity or sympathetic hyperactivity or predominance (Spallone, 2019).

Contemporary treatment by pharmaceutical or surgical means addresses symptoms but has no known effect on ANSD. Although ANSD is known to affect IHD survival and T2D prognosis, there is to date no known measure of central ANSD or evidence-based intervention which will provide reduction of (central) ANSD. ANSD is presently and often diagnosed by a combination of results of a range of tests, including Table Tilting (Novak, 2011) and Heart Rate Variability (Spallone, 2019) tests, both addressing the autonomic functions of the cardiovascular system.

Recently, it was shown that sensitivity to painful pressure of the chest bone periosteum (PPS) is a potentially useful measure of central autonomic sympathetic function, with elevated PPS measures indicating central ANSD (Faber et al 2021), linking persistent stress, depression, and IHD (Grippo and Johnson, 2009; Steptoe and Kivimäki, 2012; Staufenbiel et al., 2013; Wentworth et al., 2013), independently of other conventional risk factors (Thayer and Lane, 2007).

Central ANSD has recently been found to be reflected in elevated measures of pressure point sensitivity (PPS) at the sternum and other sympathetic tone dependent areas/points on the human body (Faber et al. 2021).

There is no evidence-based treatment of ANSD, but peripheral nerve stimulation has been suggested as a method of reduction of sympathetic nervous system hyperactivity (Güemes and Georgiou, 2018).

There is hence a need to be able to measure accurately the presence and/or severity of ANSD, and especially central ANSD to enable rational treatment that prevents negative effects caused by ANSD.

### Homeostasis and Type 2 Diabetes Mellitus (T2D)

It is known that the coordinated processes of homeostasis contribute to the maintenance of a constant condition of the interior environment in humans (Canon W B 1926; Goldstein D S 2019). The central autonomic nervous system network controls homeostasis by means of two opposing sympathetic and parasympathetic processes (Goldstein D S 2019). Levels of glycated haemoglobin (HbA1c) provide a measure of the adequacy of the autonomic homeostatic processes that regulate blood sugar concentrations and hence the energy metabolism of the body, as supervised by the orexin receptors in lateral hypothalamus (Tsuneki H et al 2012; Adeghate E et al 2020).

Previous findings by the present inventor authors formed the basis of the claim that levels of PPS reflect autonomic nervous system function (Ballegaard et 2012, Bergmann et al. 2014, Ballegaard et al. 2015, Faber et al. 2021). Studies of tilt-table reactivity and heart rate variability as measures of autonomic function, and the use of adrenergic beta-receptor blockade to test the regulatory mechanism underlying the PPS measure, suggested that the PPS is related to activation of orexin receptors in lateral hypothalamus (Ballegaard et al. 2015, 2016, Faber et al. 2021). Thus, when elevated PPS measures declined during specific non-pharmacological interventions into the well-being of healthy volunteers or patients with type 2 diabetes (T2D) with elevated HbA1c concentrations, the HbA1c measures also declined in step with the reduction of the PPS measures (Ballegaard et al. 2014, Faber et al. 2021).

Past RCT tests confirmed that PPS measures are subject to reduction by means of the specific interventions (Ballegaard et al. 2014, Bergmann et al. 2014, Faber et al. 2021). The non-pharmacological interventions have three parts:

First, daily home measurements of PPS by a simple algometric device and subsequent reflection on the values serve as indicators of daily levels of stress and tools of potential behavioural adjustment; second, daily cutaneous sensory nerve stimulations at pre-defined tender points on the body surface within specific neurological targets areas associated with the PPS measurement site serve to reduce elevated PPS measures and to maintain non-elevated PPS levels; and third, continuous professional supervision serves to manage circumstances underlying missing or deviating measurements.

### Objects of the Invention

It is an object of embodiments of the invention to provide methods for survival rate prediction and survival rate increase as well as methods for identification or evaluation of treatments of ANSD. It is also an object of the invention to provide methods for diagnosis and treatment of T2D.

### SUMMARY OF THE INVENTION

As mentioned above, there is no evidence-based treatment of ANSD, but peripheral nerve stimulation has been suggested as a method of reduction of sympathetic nervous system hyperactivity (Güemes and Georgiou, 2018). The effect of this treatment of ANSD on the survival of patients with IHD was hence tested. The treatment (the “Intervention”) consisted of a non-pharmacological program of (1) daily repeated PPS home measurements meant to induce cognitive reflection on the perceived level of stress and ANSD, and this in order to enhance compliance as well as to monitor the long-term effect, (2) daily peripheral non-noxious sensory nerve stimulation of body surface areas of specific dermatome/neurological association with the PPS measurement site at the sternum. These stimulations induce repeated autonomic reflex induced elevation of the noxious withdrawal reflex threshold for PPS reduction and thus ANSD reversal, (3) selection of free-choice physical and mental exercises, and (4) ongoing professional evaluation of Pressure Point Sensitivity (PPS) measurements with proactive supervision in cases of missing or deviating measurements (Bergmann et al., 2014).

Importantly, the association between the Intervention into elevated PPS measures with the subsequent all-cause mortality was examined. Two analyses of survival in a prospective follow-up study to the RCT reports published by Bergmann et al. (2014) and Ballegaard et al. (2015) was carried out. First, he 5-year mortalities of Intervention and Control groups were compared by Poisson regression. Second, the five-year mortalities of the two RCT groups were compared to the 5-year mortality of normal Danish populations of individuals of the same age, sex and observation period as the patients of the RCT groups.

Surprisingly, it was found that the survival rate of IHD patients associated negatively with PPS measurement values—low PPS measurement values translated into increased (prolonged) survival, whereas elevated PPS measurements translated into decreased (shortened) survival. It was also demonstrated convincingly that an Intervention treatment that can reduce elevated PPS measurements and maintain PPS measurements at a non-elevated level leads to prolonged survival.

These findings open for several practical implementations. First of all, it becomes possible to stratify IHD patients into groups having an increased death risk and a normal or even reduced death risk, respectively. In turn, this allows for rational intervention such as the one detailed herein and shown to reduce PPS measurements and increase survival, in order to increase survival rate in the IHD patients that exhibit an initial high PPS measurement value.

Also, the use of PPS measurements can be utilised to evaluate the effect on ANSD of experimental treatments (pharmacological and/or non-pharmacological), because of the PPS measurement being a surrogate value for (central) ANSD and severity thereof.

As a special case, depression in patients suffering from IHD is known to be associated with increased mortality rates, and ANSD may link the two conditions. The inventor has demonstrated that PPS at the sternum increases with increased depression in IHD, and that reduction of elevated PPS measurements reflects
1) lower depression scores, ANSD, and mortality rates in IHD, and
2) regulatory functions of the autonomic nervous system with respect to IHD risk factors including heart rate, blood pressure, serum cholesterol, and glycated haemoglobin. However, beta-adrenoreceptor blockade (BB) is found to inhibit the anti-depressive effect associated with reduced PPS measures.

It was therefore hypothesized that lowering of elevated PPS measures will be associated with enhanced autonomic regulation of depression, measured as an association between baseline depression scores and subsequent lowering of depression scores. As shown in Example 3 it can be concluded that in patients with IHD, depression is indeed associated with disruption of specific regulatory functions of the autonomic nervous system. This finding implies that lowering of elevated PPS measures indicates enhanced autonomic lowering of depression scores.

It is believed that the present findings are not limited to the IHD patient group—since ANSD is known to be associated with reduced expected survival rates in general. As ANSD is also important to many common human complaints, common diseases, severe disease such as for example T2D, and even life-threatening health conditions, which at present are treatable only by pharmaceutical or surgical means that do not address the autonomic dysfunctional aspects of the condition, the present findings are relevant and applicable to all humans, with and without disease, and for preventative and therapeutic use alike—and with no risks for side effects or complications.

Further, in a pilot study of healthy volunteers, it was noted that the non-pharmacologically induced reduction of elevated PPS measures was associated with reductions of values of blood pressure, heart rate, and serum cholesterol if, and only if, elevated at baseline. In a subsequent RCT, it was—in addition—observed that there is an association with an unpredicted reduction of levels of glycated haemoglobin (HbA1c) in healthy volunteers (Ballegaard et al 2014), all together suggesting a possible homeostatic effect of the interventions, that is indicated by the reduction of an elevated PPS.

On this basis, the inventor considered the unlikely association between central ANSD activity and measures of PPS on one hand and insufficient glycaemic homeostasis on the other, as explored by pre-defined supplementary analyses of data from the RCT (Faber et al. 2021). It was then tested (the null-hypothesis) whether reductions of elevated PPS measures at the sternum in patients with T2D by specific non-pharmacological interventions would not in principle enhance central autonomic homeostatic regulation of glucose metabolism, measured as significant reduction of HbA1c levels elevated at baseline. Surprisingly, it was found that this null-hypothesis was statistically rejected, since it was found that in persons with an elevated PPS and T2D, a reduction of the elevated PPS had a strong homeostatic effect on HbA1c measurements. The reduction of the PPS was the primary source for the obtained homeostatic effect, as demonstrated in a Responder (i.e. persons who obtained a minimum reduction of PPS during intervention period: ≥15 arbitrary units) versus Non-responder (i.e. persons who did not obtain this reduction) association analyses. Hence, central autonomic dysfunction as measured by an elevated PPS, is associated with a disrupted glucose homeostasis in people with type 2 diabetes, and amelioration of the ANSD, leads to restoration of glucose homeostasis, with a clinically relevant improved glucose metabolism. In other words, it has been found possible to diagnose (via a measure of central autonomic homeostatic regulation of blood glucose levels) and treat of T2D (by lowering an elevated PPS and subsequently lowering an elevated HbA1c and enhance autonomic homeostatic regulation of blood glucose levels).

So, in a first aspect the present invention relates to method for determining whether
1) a human subject has a lowered expected survival rate, comprising establishing, via at least one measurement of exposure to a quantifiable pain-inducing influence, the subject's pain threshold at one or more sympathetic tone dependent locations on or in the body, and determining that the human subject has a lowered expected survival rate if the subject's pain threshold is reduced compared to either a predetermined standard value or compared the subject's own pain threshold at sympathetic tone independent locations on or in the body, or
2) a human subject suffers from or is at increased risk of contracting type 2 diabetes mellitus (T2D), which can be treated via non-pharmacologic intervention, comprising establishing, via at least one measurement of exposure to a quantifiable pain-inducing influence, the subject's pain threshold at one or more sympathetic tone dependent locations on or in the body, and determining that T2D can be treated by non-pharmacological intervention, if the subject's pain threshold is reduced compared to either a predetermined standard value or compared the subject's own pain threshold at sympathetic tone independent locations on or in the body.

In a 2^{nd} aspect the present invention relates to a method of
1) increasing the life expectancy of a human subject, the method comprising determining, according to the method of any one of the preceding claims, that the subject has a lowered expected survival rate, and subsequently subjecting the subject to a treatment, which increases the subject's pain threshold a sympathetic tone dependent locations, or
2) treating a patient suffering from or having increased risk of contracting T2D, the method comprising determining, according to the method of any one of the preceding claims, that the subject has or is at increased risk of contracting T2D that can be treated via non-pharmacological intervention, and subsequently subjecting the subject to a treatment, which increases the subject's pain threshold a sympathetic tone dependent locations whereby a reduction in the HbA1c blood concentration is attained, or
3) treating a patient suffering from IHD-related depression, the method comprising determining, according to the method of any one of the preceding claims, that the subject suffering from the IHD-related depression has a lowered expected survival rate, and subsequently subjecting the subject to a treatment, which increases the subject's pain threshold a sympathetic tone dependent locations.

Finally, in closely related 3^{rd} and 4^{th} aspects the present invention relates to
a method for identifying a therapeutic treatment which has a high likelihood of reducing or curing or preventing ANSD or T2D or IHD-related depression, the method comprising via at least two measurements of exposure to a quantifiable pain-inducing influence, a determination of at least one subject's pain threshold at one or more sympathetic tone dependent locations on or in the body, subsequently identifying the treatment as one that has the high likelihood of reducing or curing preventing ANSD or T2D or IHD-related depression if the at least one subject exhibits a significant increase in the pain threshold at the one or more sympathetic tone dependent locations when receiving the treatment, and
a method for evaluating efficacy of a treatment or prophylaxis of ANSD or T2D or IHD-related depression, the method comprising via at least two measurements of exposure to a quantifiable pain-inducing influence, a determination of at least one subject's pain threshold at one or more sympathetic tone dependent locations on or in the body, wherein said at least one subject is undergoing a treatment or prophylactic treatment of ANSD or T2D or IHD-related depression, and verifying that the treatment reduces or cures or prevents ANSD or T2D or IHD-related depression if the at least one subject exhibits a significant increase in the pain threshold at the one or more sympathetic tone dependent locations.

### LEGENDS TO THE FIGURE

FIG. 1: Bar graph showing RCT populations and their 5 year death risks. Distributions of individual 5-year risks of death for the subjects of the RCT Intervention (N=106) and Control (N=107) groups. Each column represents one subject.

FIG. 2: Graph showing the accumulated number of expected 5-year deaths for the RCT Intervention and Control groups, respectively (Y-axis).

The abscissa is the magnitude of the 5-year accumulated risks for the individual subjects, shown as groups of subjects within fractions of 0.05% risk of death within the 5-year observation period matched to the general Danish population with respect to gender, age, and observation period.

FIG. 3: Graph showing distribution of probabilities of number of deaths during the 5-year observation period in two groups similar to the RCT Intervention and Control groups, respectively, when they are matched to the general Danish population for gender, age and observation period.

FIG. 4: Bar graph showing the theoretical prediction distribution of number of deaths in the RCT Intervention group based on an incidence rate of 3.3 per 1000 person-years, and the RCT Control group based on the incidence rate of 14.8 per 1000 person-years.

FIG. 5: Bar graph showing Active versus Control analysis presenting HbA1 changes in mmol/mol.

Differences in mean HbAc1 (before minus after 6 months of intervention) (mmol/mol) for three sets of patients with a predefined incremental increase in baseline HbA1c between sets are presented. Active and Control groups are presented as three blue and red columns, respectively (all between-group P<0.05) from left to the right in the order
Baseline HbA1c<53; Active,
Baseline HbA1c<53; Control,
Baseline HbA1c≥53 and HbA1c<64; Active,
Baseline HbA1c≥53; Control and HbA1c<64,
Baseline HbA1c≥64; Active,
Baseline HbA1c≥64; Control.

FIG. 6: Bar graph showing Active versus Control analysis presenting HbA1c changes in percent

Differences in mean HbAc1 (before minus after 6 months of intervention) (%) for three sets of patients with an predefined incremental increase in baseline HbA1c between sets are presented. Active and Control groups are presented as three blue and red columns, respectively (all between-group P<0.05) in the same left to right order as in FIG. 5.

FIG. 7: Bar graph showing Responders versus Non-responders analysis presenting HbA1c changes in mmol/mol:

Differences in mean HbAc1 (before minus after 6 months of intervention)(mmol/mol) for three sets of patients with a predefined incremental increase in baseline HbA1c between sets are presented. Responder groups and Non-Responders groups are presented as three green and grey columns, respectively from left to right in the order
Baseline HbA1c<53; Responders,
Baseline HbA1c<53; Non-responders,
Baseline HbA1c≥53 and HbA1c<64; Responders,
Baseline HbA1c≥53; Non-responders and HbA1c<64,
Baseline HbA1c≥64; Responders,
Baseline HbA1c≥64; Non-responders.

FIG. 8: Bar graph showing Responders versus Non-responders analysis presenting HbA1c changes in percent: Differences in mean HbAc1 (before minus after 6 months of intervention) (mmol/mol) for three sets of patients with a predefined incremental increase in baseline HbA1c between sets are presented. Responder groups and Non Responders groups are presented as three green and grey columns, respectively, in the same left to right order as in FIG. 7.

FIG. 9: Scatter plots showing the homeostatic effect measured as the correlation between changes in HbA1c during six months of intervention and baseline HbA1c for two groups: Top plot shows persons who obtain a predefined reduction of PPS≥15 during intervention period (i.e. responders) and bottom plot shows persons, who do not obtain this reduction (Non-responders). Responders=1, non-responders=0).

### DETAILED DISCLOSURE OF THE INVENTION

### Definitions

The expression “exposure to a quantifiable pain-inducing influence” denotes any type of exposure of a human to an influence, which activates the skin's mechanoreceptors, thermoreceptor and/or nociceptive receptors in a non-binary manner (meaning that the person exposed to the influence can sense a gradual increase in the exposure to the influence from a non-painful level up to the level of the pain threshold). The fact that the pain-inducing influence is quantifiable has the consequence that it is possible to measure quantitively the amount/intensity of the influence and correlate it to the human's indication of the exposure, which is painful. Exposure may be provided as mechanical, thermal, radiation, electric, and/or chemical stimuli, preferably mechanical. A mechanical stimulation may for instance be provided by means of a compressive force. A thermal stimulation may for instance be provided by means of cold and/or heat, preferably increase in heat. Radiation stimulation may for instance be provided by means of an applied infrared, visible and/or ultraviolet light or combined spectra thereof, e.g. a laser, light-emitting diode, infrared, ultraviolet and/or white light source. Chemical stimulation may be provided by means of an organic and/or an inorganic compound. Electric stimulation can be accomplished by applying both AC and DC currents.

The expression “sympathetic tone” denotes the level of activity in the sympathetic part of the autonomic nervous system and is a measure of a person's potential to perform optimally both physically and mentally.

The expression “sympathetic tone independent location” denotes a location on the body in which the sensitivity to an exposure of the quantifiable pain-inducing influence is independent of the activity level of the sympathetic nervous system. Also covered by the expression is a point on the body where increased sympathetic tone causes a higher threshold for sensitivity or nociception in said point.

The expression “sympathetic tone dependent location” denotes a location on the body in which the sensitivity to an exposure of the quantifiable pain-inducing influence is dependent on the activity level of the sympathetic nervous system, in the sense that increased sympathetic tone causes the location to exhibit a lowered pain threshold. Several such locations exist on the body and are e.g. detailed in WO 2005/084529, WO 2006/092146, and WO 2008/028976; the preferred location is over the periosteum covering the anterior surface the sternum, more precisely the most pain-sensitive area when applying pressure to the skin covering the anterior surface of the sternum.

A “significant increase” or “significant decrease” in pain threshold is a significant deviation from the established normal pain threshold. This can either be determined individually, where the pain threshold in sympathetic tone dependent locations is compared to the pain threshold of sympathetic tone independent locations in the same human subject. Alternatively, the significant increase/decrease can be established against a population average. In both cases, a significant decrease or increase must be outside a selected confidence interval surrounding an established mean, e.g. outside the 95% confidence interval.

The expression “IHD-related depression” indicates the presence of both clinical depression (i.e. a major depression inventory (MDI) score>=15, or equivalent when other measures for depression score are used) and IHD in a patient, irrespective of the causative relationship between the two disease conditions. For details about MDI scores, cf. Bech P, “Clinical Psychometrics”; Oxford: Wiley Blackwell; 2012; see also Bech P et al. (2015), BMC Psychiatry 15:190 (doi: 10.1186/s12888-015-0529-3).

### Specific Embodiments of the Invention

### Embodiments of the 1^{st} Aspect of the Invention

The 1^{st} aspect relates to a method for determining whether
1) a human subject has a lowered expected survival rate, comprising establishing, via at least one measurement of exposure to a quantifiable pain-inducing influence, the subject's pain threshold at one or more sympathetic tone dependent locations on or in the body, and determining that the human subject has a lowered expected survival rate if the subject's pain threshold is reduced compared to either a predetermined standard value or compared the subject own pain threshold at sympathetic tone independent locations on or in the body, or
2) a human subject suffers or from or is at increased risk of contracting type 2 diabetes mellitus (T2D), which can be treated via non-pharmacologic intervention, comprising establishing, via at least one measurement of exposure to a quantifiable pain-inducing influence, the subject's pain threshold at one or more sympathetic tone dependent locations on or in the body, and determining that the T2D can be treated by non-pharmacological intervention, if the subject's pain threshold is reduced compared to either a predetermined standard value or compared the subject own pain threshold at sympathetic tone independent locations on or in the body. As explained above, the measurement of subject's pain threshold at one or more sympathetic tone dependent locations on or in the body provides for a surrogate measure of life expectancy, and also of the degree of ANSD or the curability of T2D in subjects suffering from this condition.

Determination of the pain threshold (nociception threshold) in sympathetic tone dependent locations as a means of determining sympathetic tone, stress levels, and mental fitness is the subject of WO 2005/084529, WO 2006/092146, and WO 2008/028976. These patent application publications disclose devices and methods for nociception threshold determination in humans and their application in determination of sympathetic tone status, stress status and mental fitness. In preferred embodiments of the 1^{st} aspect of the invention, any disclosure in these three references, which relate to pain threshold determination and evaluation thereof for the purposes of determining whether any pain threshold in a sympathetic tone dependent location is reduced vs. population levels or vs. pain threshold in sympathetic tone independent locations, applies mutatis mutandis to the present method of the 1^{st} aspect, and also to the further aspects of the present invention.

Hence it is within the scope of the 1^{st} aspect of the invention that reduction in pain threshold at the sympathetic tone dependent location is reflected in a corresponding reduction in the measurement value of the exposure to the quantifiable pain-inducing influence. As an example, if a subject initially exhibits a reduced threshold for pain caused by mechanical pressure exerted on the frontal face of the sternum, this is reflected in a reduced force necessary to induce pain with the device disclosed in 2005/084529 and WO 2006/092146, when comparing to either the force necessary to induce pain in a sympathetic tone independent location or when comparing to pain sensitivity in the healthy population.

In case of option 2, and as shown in the examples, the lowered pain threshold (i.e. increased pain sensitivity) is typically accompanied by an elevated HbA1c blood concentration, so in some embodiments, it is also of value to concentrate on those patients that exhibit such an increase in order to identify those that have T2D that can be treated by non-pharmacologic interventions as those disclosed herein. Similarly for healthy persons who have elevated HbA1c (e.g. HbA1c≥31 Mmol/mol) (Ballegaard et al. 2014) As mentioned above, the quantifiable pain-inducing influence can take any of a plurality of forms, but it is preferably selected from heat, localized physical pressure, and electricity, and in particular localized physical pressure; whereas chemical stimuli are not excluded as a possibility, it is less convenient to apply chemical stimuli quantitatively in a manner allows a continuous transition from non-pain inducing exposure to pain inducing exposure.

In line with the above, the measurement of exposure to the quantifiable pain-inducing influence normally comprises at least one measurement of pressure point sensitivity (PPS), i.e. at least one measurement of increasing localized mechanical pressure up to the pain-inducing exposure.

The present method of the 1^{st} aspect, option 1, finds particular utility when the human subject a priori has a lowered expected survival rate due to an illness. That is, if the human subject has a disease or condition, which generally is associated with lowered expected survival rate, the method of the present invention allow for a determination which can verify or disprove that the human subject has a lowered expected survival rate, which in turn opens for the possibility of focusing life expectancy increasing measures in those human subjects that are verified as having the lowered survival rate. Likewise, the approach can also be applied in subjects that according to known data have a normal or even increased survival rate in order to identify those who in reality have a reduced expected survival rate and then devise a treatment/preventive regimen for these subjects.

If concentrating the method of the first aspect on subjects having an a priori reduced expected survival rate, the illness is preferably autoimmune diseases in general, diabetes mellitus (type I or type II), cancer, or cardiovascular disease, in particular ischemic heart disease (IHD). Also of particular interest are patients that have an a priori reduced expected survival rate because they have IHD-related depression.

As mentioned above, the any of the one or more sympathetic tone dependent locations discussed in WO 2005/084529, WO 2006/092146, and WO 2008/028976 are useful in the practice of the 1^{st} aspect of the invention, but it is for practical reasons preferred that the location is or comprises the periosteum of the anterior part of the sternum.

### 2^{nd} Aspect of the Invention

The aspect relates to a method of
1) increasing the life expectancy of a human subject, the method comprising determining, according to the method of the 1^{st} aspect of the invention and any embodiment thereof disclosed herein, that the subject has a lowered expected survival rate, and subsequently subjecting the subject to a treatment, which increases the subject's pain threshold at sympathetic tone dependent locations, or
2) treating a patient suffering from or having increased risk of attracting DT2, the method comprising determining, according to the method of any one of the preceding claims, that the subject has or is at increased risk of contracting T2D that can be treated via non-pharmacological intervention, and subsequently subjecting the subject to a treatment, which increases the subject's pain threshold at sympathetic tone dependent locations whereby a reduction in the HbA1c blood concentration is attained, or
3) treating a patient suffering from IHD-related depression, the method comprising determining, according to the method of any one of the preceding claims, that the subject suffering from the IHD-related depression has a lowered expected survival rate, and subsequently subjecting the subject to a treatment, which increases the subject's pain threshold a sympathetic tone dependent locations.

The term treatment must be understood in a broad sense and includes both therapeutic approaches (pharmacological as well as non-pharmacological) and approaches that aim at changing lifestyle in a manner that facilitates an increase in the measurements of pain thresholds, which generally serve as a surrogate measure of life expectancy.

Preferably, the treatment's efficacy will be verified by repeatedly measuring the subject's pain threshold according to the method of the 1^{st} aspect, by ensuring that the pain threshold is reduced as evidenced by increase over time of measurement values of the quantifiable pain-inducing influence. These measurements can be performed by the subject itself, e.g. at home.

In preferred embodiments, the human subject subjected to the method of the 2^{nd} aspect is a subject who a priori has a lowered expected survival rate due to an illness as discussed in detail under the 1^{st} aspect, but the subject can also be one who does not a priori have a lowered expected survival rate; the latter is in line with the discussion above of identifying persons who in spite of an a priori normal or high life expectancy are found to have a reduced life expectancy.

The treatment preferably comprises repeated, such as daily, autonomic reflex activation and/or personal empowerment. Repeated autonomic reflex activation may for instance comprise peripheral non-noxious sensory nerve stimulations at a sympathetic tone dependent point, preferably involving the anterior sternal periosteum, or any other locations within the spinal segmental innervation area or with a relevant neurological association of the relevant measurement site or other locations outside these areas with a neurological connection to the measurement site, for repeated autonomic reflex elevation of the noxious withdrawal reflex threshold, and wherein the personal empowerment comprises free-choice physical and mental exercises. Cf. the Example below for more details on one suitable treatment option

In particularly preferred embodiments, the method of the 2^{nd} aspect comprises repeated (home) PPS measurements and optionally ongoing PPS measurement evaluation that addresses missing or deviating PPS measurements; this embodiment is hence in line with the methodologies taught in WO 2005/084529, WO 2006/092146, and WO 2008/028976.

A variation of the 2^{nd} aspect method, option 1, is to ensure—in subjects who do not exhibit a lowered life expectance when subjected to the method of the 1^{st} aspect and embodiments thereof—that these subjects maintain their non-lowered life expectancy. This may be achieved with substantially the same types of intervention and repeated measurements set forth above, but instead of seeking to increase life expectancy, the method seeks to identify any subsequent signs of changes in the pain sensitivity that could signal that the life expectancy is beginning to drop. Similar considerations apply to persons not suffering from T2D, but where signs of future development of TD2 can be identified.

### 3^{rd} and 4^{th} Aspects of the Invention

These aspects are closely related and constitute:
a method for identifying a therapeutic treatment which has a high likelihood of reducing or curing (central) or preventing ANSD or T2D or IHD-related depression, the method comprising via at least two measurements of exposure to a quantifiable pain-inducing influence, a determination of at least one subject's pain threshold at one or more sympathetic tone dependent locations on or in the body, subsequently identifying the treatment as one that has the high likelihood of reducing or curing or preventing ANSD or T2D or IHD-related depression if the at least one subject, when receiving the treatment, exhibits a significant increase in the pain threshold at the one or more sympathetic tone dependent locations, and
a method for evaluating efficacy of a treatment or prophylactic treatment of (central) ANSD or T2D or IHD-related depression, the method comprising via at least two measurements of exposure to a quantifiable pain-inducing influence, a determination of at least one subject's pain threshold at one or more sympathetic tone dependent locations on or in the body, wherein said at least one subject is undergoing a treatment or prophylactic treatment of ANSD or T2D or IHD-related depression, and verifying that the treatment reduces or cures or prevents ANSD or T2D or IHD-related depression if the at least one subject exhibits a significant increase in the pain threshold at the one or more sympathetic tone dependent locations.

Both these methods take advantage of the finding made herein, namely that pain threshold measurements provide for a surrogate measure of both not only life expectancy but also of ANS function, hereunder of the presence of ANSD and thus potentially disrupted homeostasis. This surprising finding opens the door for a rational way of evaluating whether a therapy, such as administration of a drug, is capable of reducing ANSD or even normalizing AND function to arrive at the desired balanced state of homeostasis

In preferred embodiments of both the 3^{rd} and 4^{th} aspects, the at least one subject at the onset of the method exhibits a reduced pain threshold at the one or more sympathetic tone dependent locations. Put in a different language, it is by involving subjects that have lowered pain threshold (signifying the presence of ANSD) possible to directly measure whether the treatment evaluated has the desired effect, whereas it could be difficult in the event the subjects already have desired/normal ANS function.

In both of aspects 3 and 4 it is preferred that the at least one subject's pain threshold is determined repeatedly, such as daily, every second day, or weekly.

In the most preferred embodiments of the 3^{rd} and 4^{th} aspects, the at least one measurement is carried out as discussed above under the 1^{st} aspect of the invention and any embodiments thereof disclosed herein.

For the 3^{rd} aspect, the at least one subject is typically a plurality of subjects in order for the identification of the therapeutic treatment to be based on statistically significant findings in a group of subjects. Also, the at least one subject should normally be one who suffers from ANSD or T2D or IHD-related depression.

### Example 1

### Clinical Study of ANSD Impact on Survival Rate in IHD Patients

### Objective, Design, Participants, and Setting

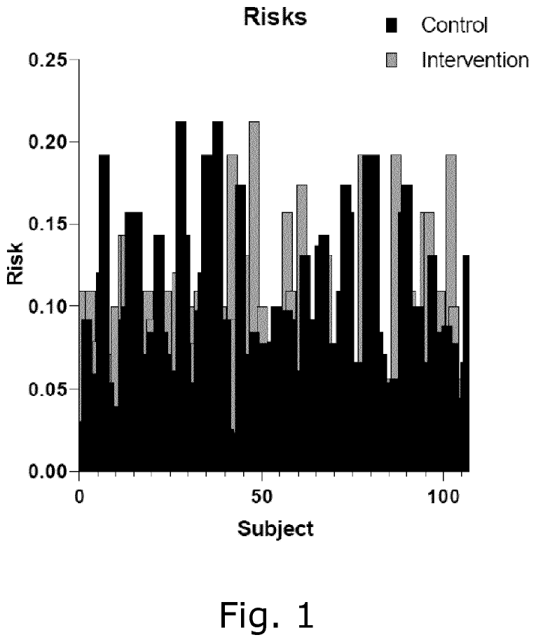

The null hypothesis was tested that reduction of elevated PPS measures during a three-month program of non-pharmacological Intervention would not reduce the five-year mortality of patients with chronic but stable IHD. The test was designed as a follow-up to the previously reported three-month randomized controlled trial (RCT) of 213 patients (Bergmann et al., 2014; Ballegaard et al. 2015). The choice of five years of observation made use of five-year intervals of data from Statistics Denmark of the general Danish population, matched for gender, age, and observation period. Five-year all-cause survival of the patients of the Intervention group was compared to the all-cause survival of the group of Control subjects, and separately both of these groups were compared to matching versions culled from the data of the general Danish population, matched for age, gender, and observation period. The cohorts of the original RCT trial consisted of 106 actively treated patients (i.e., subjects receiving the Intervention designed to reduce elevated PPS measures), and 107 Control subjects (FIG. 1). Mean age at inclusion was 62.7 (±8.1 SD) years, with 73% males and 27% females. The calendar years of inclusion into the study were 2011/2012 for 80/26 patients of the Intervention group, and 79/28 of the Control group, respectively. No contact was made between subjects and research team after the three months of RCT.

### Outcome Measure

Healthcare data for Denmark is available for anonymized research via Statistics Denmark and the Central Population Register provides information on vital status and all-cause mortality in Denmark. The outcome measure was all-cause mortality, as obtained from this register. Due to General Data Protection Regulations of Statistics Denmark, any number of clinical events less than or equal to three used in statistical analysis must be presented as “three or fewer events”. The present study complies with this regulation.

### Intervention and Control Groups

Results of the previous three-month Intervention were reported in detail (Bergmann et al., 2014; Ballegaard et al., 2015). Members of the RCT Intervention group completed the three-month non-pharmacological, self-care stress reduction program, including home use of a PPS measuring device, with instruction to perform daily home PPS measurements and sensory nerve stimulations designed to reduce the PPS measurements. During the three months, professional backup was conducted by ongoing on-line monitoring of the PPS measures, with provision for a professional instructor to act proactively in case of missing or deviating measures. Patients of the RCT Control group received the information that the level of persistent stress was elevated and unfavourable for the disease prospects, as well as a book written by an acknowledged medical stress specialist that underscored the association and gave advice for general stress management. The programs of the two groups included no medication other than the prescriptions active at the beginning of the study, and the medications remained unchanged during the last one month prior to onset of, as well as during, the three-month study periods. At the end of the three-month study period, patients of the RCT Intervention group were encouraged to continue the daily efforts with the goal of maintaining low PPS, but degree of compliance, if any, was not registered.

### Statistics

Via the unique 10-digit central person registry (CPR) number, all persons in Denmark can be tracked with respect to mortality. Three all-cause mortality analyses (for statistical details) were carried out. One analysis compared the all-cause mortalities of the Intervention and Control groups of subjects of the RCT on an intention-to-treat basis. Poisson regression of rare outcome events with SAS Statistical Software package version 9.4 (SAS Institute Inc., Gary, NC, USA) and Stata Software version 15 (StataCorp, College Station, TX, USA) was employed. The additional analyses compared the all-cause mortality of each of the two groups of the RCT with subsets of the general Danish population, matched for age, gender, and observation period. Statistics Denmark delivers all-cause mortality data for five-year intervals, allowing us to compare each of the 213 patients of the RCT to approximately 35.000 Danes with the same age and gender for the five-year observation period, each starting at the time of the entry into the original RCT study (Bergmann et al. 2014).

The distribution of deaths in subgroups of persons from the Danish general population was predicted based on data from Statistics Denmark with the same age and gender profiles of each individual patient of the RCT (Bergmann et al. 2014). While a one-sided t-test is consistent with the null hypothesis that mortality would not decline in the Intervention group, results of both one-sided and two-sided t-tests (Cox et al., 1977) are presented.

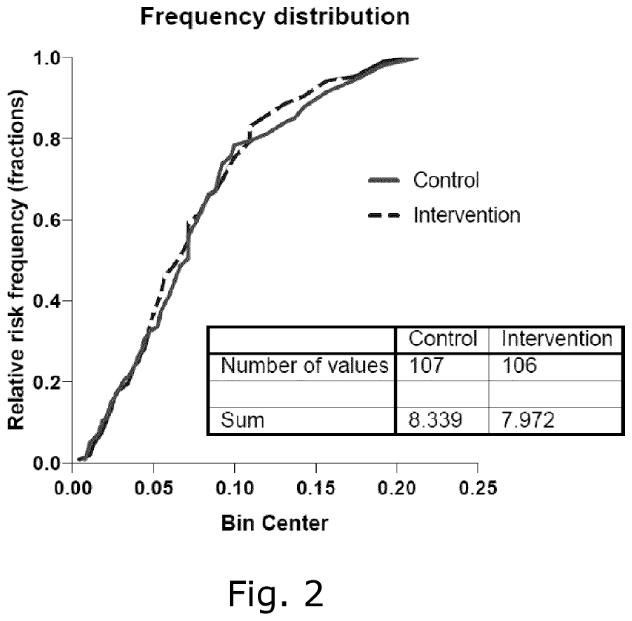

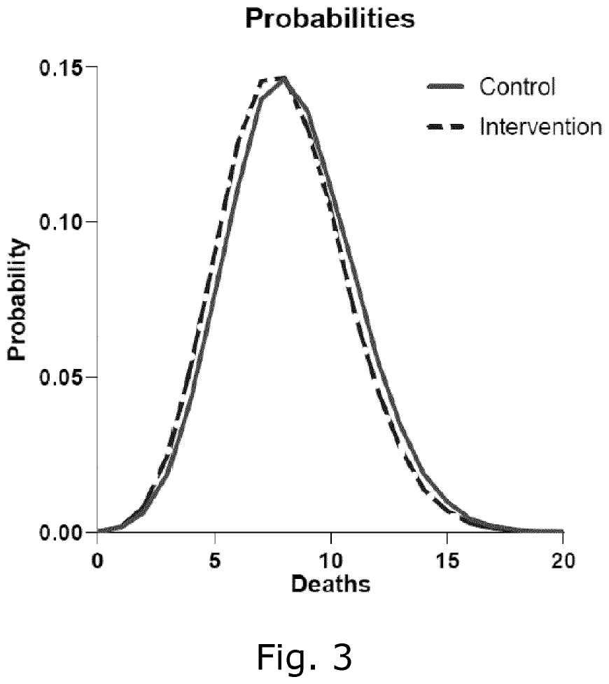

The distribution of the individual five-year risk of death of the 106 persons of the RCT Intervention group and the 107 subjects of the RCT Control group is non-symmetrical (FIG. 1). Based on the background population of Denmark, the distribution of the number of deaths in a group was estimated to be similar to the Intervention and in a group similar to the Control group, considering the individual risks. The two groups were similar with respect to the predicted number of deaths of 7.97% and 8.34% for the Intervention and Control groups, respectively (FIGS. 2 and 3).

### Results

### Intervention Versus Control Groups

During the years 2011-2016, 11 or fewer deaths were observed during the median period of observation of 5.2 years, including three or fewer subjects from the Intervention group, and eight subjects from the Control group. At baseline, 20 patients of the Intervention group and eight patients of the Control group had Type 2 Diabetes Mellitus (between group P=0.027). Adjusting for this difference, Poisson regression of survival data from the participants of the RCT trial disclosed a significant difference of survival between the subjects of the Intervention and Control groups (one sided t-test, P=0.017; two-sided t-test, P=0.035). All-cause mortality was lower for the Intervention group with incidence rates (IR) of 3.7 per 1000 person-years, and adjusted incidence rate ratios (IRRs) of 0.19 (95% confidence interval: 0.04-0.93), compared with Control subjects (IR 14.8 per 1000 person-years).

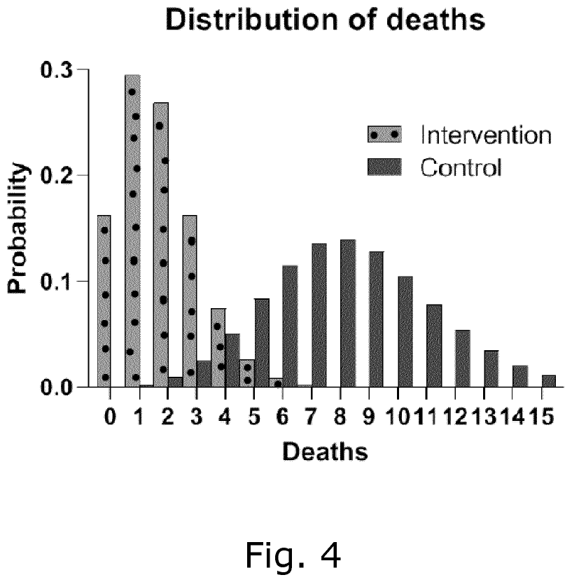

As shown in FIG. 4, a mean prediction of 1.81896 deaths derived from the Intervention mean of 3.33 deaths per 1000 person-years (py), obtained by applying 3.33 per 1000 py to the experiment of 106×5.2 py where 106 is the number of subjects and 5.2 the number of years followed, i.e., 3.3×(106×5.2/1000)=1.81896. The period of 5.2 years is the median, as half of the subjects were followed for less than 5.2 years, and half were followed for more than 5.2 years. The prediction of 1.81896 years is therefore the Poisson mean that applies when all 106 participants were followed for exactly 5.2 years. Similarly for the Control group, the theoretical mean prediction of 8.23472 deaths derived from the Control group mean of 14.8 deaths per 1000 person-years (py), obtained by applying the 14.8 per 1000 py to an experiment of 107×5.2 py where 107 is the number of subjects and 5.2 the number of years followed, i.e., 14.8×(107×5.2/1000)=8.23472.

### Intervention and Control Groups Versus General Population of Denmark

Table 1 shows the risks of death, the expected number of deaths as well as the observed number of deaths, within the five-year observation period of members of the Intervention and Control groups of the RCT, matched to subjects from the general Danish population with the same age and gender, according to Statistics Denmark. When compared to the expected number of deaths during the five-year observation period, the number of three or fewer deaths of the Intervention group was significantly lower than the expected number of eight deaths (one-sided t-test, P=0.010; two-sided t-test, P=0.043, while the observation of eight deaths in the Control group matched the prediction (P=0.54).

TABLE 1
Distribution of five-year risks of death (range and mean), predicted
number of deaths in the Intervention and Control group of the
RCT when matched to general Danish populations for age, gender
and observation period, and observed number of deaths
(C.I = confidence limits; N.S: = non significant).

| | | | Predicted | Number | |
|---|---|---|---|---|---|
| | | Range | no. of | of deaths | |
| RCT group | n | (mean) | deaths | (95% C.I.) | P-value |
| Intervention | 106 | 0.0043-0.2123 | 7.97 | ≤3 | 0.01 |
| group | | (0.075) | | (0.5-6.6) | |
| Control | 107 | 0.0076-0.2123 | 8.33 | 8 | N.S. |
| group | | (0.078) | | (3.8-14.1) | |

### Discussion

### Main Findings

The present RCT tested survival after a specific hypothetical adjunct Intervention into ANSD in IHD, claimed to ameliorate an assumed dysfunctional cerebral regulation of autonomic function, expressed as pathologically elevated measures of PPS. The original RCT was carried out as a single-center, two-armed, parallel-group, observer-blinded, randomized (1:1), clinical superiority trial, and subsequently tested the effect of the non-pharmacological intervention in terms of the subsequent five-year all-cause mortality. The Intervention was designed to lower the elevated PPS reading at the periosteum of the sternum as a measure of ANSD. The findings rejected the null hypothesis that three months of PPS-guided non-pharmacological adjunct Intervention would not lower the five-year all-cause mortality of patients with IHD and elevated PPS measures above 60 arbitrary units at baseline.

In previous RCT of IHD patients, the Intervention lowered the questionnaire ratings of depression reported by the patients (Bergmann et al., 2014), and lower depression ratings are known to be associated with lower incidence of all-cause mortality in IHD patients (Lichtman et al. 2014) that in turn has been linked to lower ANSD ratings (Grippo and Johnson, 2009). The Intervention also improved measures of systolic blood pressure response to tilt-table testing (Ballegaard et al., 2015), also known to be associated with lower levels of ANSD. Both effects were reduced in the presence of beta-adrenergic medication (Ballegaard et al., 2016), consistent with the hypothesis advanced by Bergmann et al. (2014) and Ballegaard et al. (2015) that reduction of the degree of ANSD, measured as a reduction of an elevated PPS, is due to lower autonomic sympathetic activity, a notion which is further supported by the association between PPS and sympathetic cardiovascular neuropathy in persons with type 2 diabetes (Faber et al. 2021).

Second, five-year all-cause mortalities of the two RCT groups was compared to subsections of the general Danish population, matched for gender, age, and observation periods. The findings rejected the null hypothesis that three months of adjunct Intervention would not lower the five-year all-cause mortality when compared to the general Danish population matched for gender, age and observation period. However, the findings did not reject the null hypothesis that the five-year all-cause mortality of the RCT Control group would equal that of the general Danish population, matched for gender, age, and observation period.

Thus, the present findings are not consistent with the null hypothesis that three months of PPS-guided non-pharmacological adjunct Intervention would not lower the five-year all-cause mortality of IHD patients with elevated PPS. The subsequent evaluation of possible other causes of the effect of the Intervention revealed no significant effect other than the lowering of elevated PPS values, interpreted as indicative of attenuation of central ANSD.

As the inclusion criterion of participation was ANSD at baseline, defined as an elevated PPS measure of at least 60 units, the findings underscore the association between ANSD indicative of increased sympathetic autonomic brain activity measured by PPS, and the prognostic association with all-cause mortality. The decreased mortality of the Intervention group compared to the general population underscores the association between PPS measures and central autonomic function.

### Modulation of Sensitivity to Painful Pressure

The afferent leg of the neurophysiological pain loop can be modulated by non-pharmacological sensory nerve stimulation in patients with painful diabetic neuropathy (Tesfaye et al., 2010), as well as by activity of efferent pathways from cerebral neurotransmission, and by receptor blockade in the spinal cord (Richner et al., 2019). The reduction of the PPS measure achieved by the present Intervention appears to be mediated by a central reflex loop (Faber et al. 2021). Furthermore, non-painful cutaneous sensory nerve stimulation releases the hypothalamic peptide oxytocin that reduces pain and stress measures, restores autonomic function, and engages beta-adrenergic receptors (Uvnäs-Moberg et al., 2014). The evidence suggests an association of the PPS measure to the orexin receptor system of the lateral hypothalamus rather than the oxytocin system (Faber et al. 2021).

PPS Measure, ANSD, and Mortality in Patients with Ischemic Heart Disease

It is well established that ANSD is associated with increased mortality in IHD (Thayer et al., 2007). The association between PPS and central ANSD has been established by a series of studies, including tilt table response in IHD (Ballegaard et al., 2015, 2016) and cardiovascular neuropathy in diabetes patients (Faber et al., 2021). Despite substantial improvements of the survival of patients with ischemic heart disease, the patients still face an elevated risk compared to the general population (Sidney et al., 2018), especially if the patients have type 2 diabetes mellitus (Quinones et al., 2015). Given that all patients of the original RCT had ischemic heart disease, and that a substantial number had diabetes as well, it is of considerable interest that the mortality of the Intervention group was found to be significantly lower than that of the general Danish population.

It cannot be ruled out that a measure of biofeedback, other than the PPS measures, would similarly be indicative of an effect on all-cause mortality. However, in the available literature of RCT of IHD, no evidence was found of reduction of any individual cardiovascular risk factor that lowered all-cause mortality to a level that surpassed that of the general population, matched for gender, age and observation period (Sidney et al., 2018). The lack of such evidence supports the claim that reduction of central ANSD is achieved by the Intervention, as monitored by the reduction of the elevated PPS. The PPS reading is interpreted as a measure of function of the ANS that originates at levels of the ANS hierarchy higher than those of other individual risk factors associated with IHD (Faber et al., 2021).

To be sure, in a series of RCT, reduction of elevated PPS measures was found to be associated with reduction of a broad range of independent health risk factors that are known to affect mortality, including autonomic dysfunction measured as the tilt table test response (Ballegaard et al., 2015), as well as blood pressure, heart rate, work of the heart measured as the Pressure-Rate Product (i.e., systolic blood pressure×heart rate), serum lipids, glycated hemoglobin A1c, body mass index, low-grade inflammation (Ballegaard et al., 2014), depression, stress and general health measured by questionnaires (Bergmann et al., 2014). It is noted that the health risk factors of this list are all affected by changes of the autonomic nervous system as recorded by PPS measures (Ballegaard et al., 2014, 2015), and autonomic nervous system dysfunction has been linked to effects of these factors, including impaired pain control (Chalaye et al., 2012), cardiovascular disease, depression, persistent stress (Grippo and Johnson, 2009; Steptoe and Kivimäki, 2012), metabolic syndrome, i.e., hypertension, disturbed lipid and glucose metabolic rates with disturbed body fat distribution (Rosmond, 2005), and low-grade inflammation (Bhati et al., 2019). Taking these findings into account, the present evidence does not reject the claim that the PPS measure reflects a central ANS function that affects the risk factors (Faber et al., 2021).

The null hypothesis was based on the following potentially confounding factors, all of which have been evaluated for possible effect on all-cause mortality in IHD patients, with a negative result. Physical exercise rehabilitation has been found to reduce cardiovascular mortality, but not all-cause mortality, implying that death from other causes may occur earlier (Anderson et al., 2016). Both reduction and gain of weight have been found to increase all-cause mortality (Dong et al., 2018). Yoga-based cardiac rehabilitation programs do not affect the rate of cardiac events or all-cause mortality (Prabhakaran et al., 2020). A recent Cochrane review (Richards et al., 2018) evaluated the effect of psychological intervention, relaxation therapy, cognitive behavioural therapy, mindfulness, transcendental meditation, health education, stress management and life style programming, tele-health intervention, and psychotherapy and revealed no effect on all-cause mortality.

Furthermore, cognitive therapy towards low perceived social support has been found not to lower all-cause survival, or the rate of a new cardiac event (Schneiderman et al., 2004), and although self-reported measures of religiosity and spirituality have been reported to affect all-cause mortality in healthy persons, no studies evaluated these activities as forms of secondary intervention in IHD (Koenig, 2012). With respect to diets, including Mediterranean diet, the authors of a recent Cochrane review concluded that there is a paucity of evidence for effects of secondary intervention into IHD (Rees et al., 2019). In other Cochrane reviews, the authors examined the effect of dietary reduction of saturated fat or Omega 3 fatty acids and found no significant effect on all-cause or cardiovascular mortality (Abdelhamid et al., 2018; Hooper et al., 2020).

Thus, none of the potential confounders that patients may have experienced during the five-year observation period have been found to reduce the all-cause mortality of IHD patients to a level that surpasses that of the general population, matched for gender, age, and observation period or to reduce all-cause mortality in IHD. Other possible explanations of the reduction of all-cause mortality have not been identified, leaving the reduction of central ANSD as plausible explanation of the lowered mortality.

### Considerations

Access to mortality data is a well-documented and validated application of information from Statistics Denmark. The rejection of the null hypothesis is interpreted to mean that ANSD was reversed by the Intervention that included repeated autonomic reflex activation and personal empowerment, and that normal ANS function would be maintained unless or until a new and severe strain would cause a recurrence of ANSD. Furthermore, the findings are interpreted to suggest that the subjects of the Intervention continued measuring the PPS at the end of the three months of RCT (Bergmann et al., 2014), for continued personal reflection on the level of stress, and treatment of self by sensory nerve stimulation to maintain non-elevated PPS values by subjects aware of the possible benefit. However, the results of mortality were collected during the subsequent five years from nationwide registers, with no further contact between patient and research team, in order to observe the natural course after the Intervention and to exclude potential influence from researcher bias.

It is a challenge to the statistics that only a small number of deaths was observed. It was expected that the statistical tests of the effect would have low power and reflect a high risk of Type II errors. For this reason, the effect of Intervention on mortality was tested in three ways. Poisson regression was chosen because of the small number of events and the mortalities of the participants of the Intervention and Control groups were compared with those of the subgroups of the general population. Regardless of the expected low power of the statistical tests, the results of the three analyses were consistent with the rejection of the null hypothesis. Furthermore, the combination of a statistically significant reduction in mortality and the expected low power of the statistical tests increased the strength of the evidence supporting the conclusions, as small and inconsequential differences in mortality would not have resulted in significant test results. It was found that the five-year risk of death of the RCT Control group corresponds to the five-year risk of death in the matched normal population, although a slightly increased risk was to be expected as all the participants had chronic ischemic heart disease and some in addition type-2 diabetes (Quinones et al., 2015; Sidney et al., 2018). However, the normal risk may be explained by the RCT Control group's active interaction with the researchers that could have affected both the level of stress (Bergmann et al., 2014) and the level of autonomic dysfunction in a beneficial way (Ballegaard et al., 2015).

Tests of hypotheses involve more than checks of whether a P-value is lower than 5%. When the research hypothesis is a historical null-hypothesis, the magnitude of the P-value is a measure of how strongly the statistical test rejects the hypothesis, with lower P-values providing stronger rejection. In this case, the null hypothesis claimed that the intervention would fail to reduce all-cause mortality. The rejection is supported by clinical experience and relevant risk factor reductions (Ballegaard et al., 2014, 2015; Bergmann et al., 2014), as well as on two other prospective observational studies, in which it was specifically concluded that the results (i.e., reduced overall mortality observed of an non-pharmacological PPS-reducing intervention when compared to that of the general Danish population matched for gender, age, and observation period) would only be valid if tested by a strict RCT design (Bergmann et al., 2014, Ballegaard et al., 2015). In this respect, the present conclusions are consistent with the significance from one-sided t-tests, and the restricted presentation of the number of deaths does not affect the significance or the confidence intervals. However, the rejection was also significant when tested by two-sided statistics. Finally, large sample sizes are preferable also for evaluation of potential side effects. However, this is of less concern here because previous studies showed that the present intervention carried neither risks nor side effects (Bergmann et al., 2014; Ballegaard et al., 2015).

### Conclusions and Possible Clinical Applications and Perspectives

The present results of reduced mortality after the Intervention add to previous reports of evidence of benefits. They also imply that the proposed Intervention into autonomic dysfunction and ischemic heart disease potentially is applicable as a supplement to conventional treatment of ischemic heart disease. Central autonomic dysfunction is a key to the understanding of cardiovascular pathology and thus to IHD that may not be fully addressed by present pharmaceutical and surgical means. The PPS tool and the associated treatment are applicable to assessment of central autonomic dysfunction in patients with IHD for therapeutic and preventive purposes, and with no risk of side effects. Autonomic dysfunction is also important to many common human complaints, common diseases, and even life-threatening health conditions, at present treatable only by pharmaceutical or surgical means that do not address the autonomic dysfunctional aspects of the condition. Accordingly the present measurement method and associated intervention may be applicable in these conditions as well, for therapeutic as well as for preventative purposes.

### Example 2

### Intervention Effect on PPS and HbA1c

### Background

Living organisms maintain a constant interior environment by means of coordinated processes of homeostasis, controlled by the autonomic nervous system. It was tested whether normalization of elevated measures of Pressure Pain Sensitivity (PPS) at the chest bone by non-pharmacological means in patients with Type 2 Diabetes (T2D) could be associated with renewed glucose metabolic homeostasis, as expressed by normalization of elevated HbA1c measures in blood plasma.

### Study Design

In a randomized controlled trial of 144 patients with T2D, the hypothesis that reduction of elevated PPS in T2D would be inconsistent with reduced levels of HbA1c in blood plasma was tested. The homeostatic effect (i.e. the increase in reduction of HbA1c in response to intervention, when baseline HbA1c increases) was compared by regression analysis of reduction in HbA1c versus baseline HbA1c of persons with a pre-defined minimum reduction of PPS (i.e. ≥15 arbitrary units: “responders”, N=52) against the effect in persons who did not obtain this effect (i.e., “non-responders”, N=60).

The present study was a prespecified sub-study of a randomized clinical trial (RCT). As such the study population was divided by stratification during the randomization process in three groups, according to international standard based on an incremental increase in HbA1c at baseline; Group A: HbA1c<53 mmol/mol; Group B: 64 mmol/mol>HbA1c≥53 mmol/mol; and Group C: HbA1c≥64 mmol/mol (Faber et al 2021).

Advantage was taken by the fact that the treatments were non-pharmacological. This enabled a two set of analyses: Active versus Control and Responders versus Non-Responders.

As the Control group received an active intervention as well, and as the hypothesis of the study refers to the reduction of an elevated PPS rather than the effect of the specific active treatment, the data from the Active and Control groups are pooled and divided with respect to the size of the reduction of the elevated PPS at baseline. As such, the minimal clinical relevant reduction of the elevated PPS over 6 months is predefined as 15 PPS arbitrary units. Persons, who obtain this reduction are designated “Responders”, while persons who do not obtain this reduction are designated “Non-responders”.

The effected variable were: Glycated hemoglobin (HbA1c) and PPS.

### Analyses Derived on the Basis of the Primary Effect Variable:

### Measurement of Homeostatic Effect

With respect to the difference between Active and Control treatment, this allowed to adjust for the “regression towards the mean” as this effect was represented in the Control group. By comparing the correlation coefficient and regression line between baseline HbA1c versus changes in HbA1c during the intervention period, these provide a measure for the homeostatic effect.

From a clinical perspective, the reduction of the elevated HbA1c for three groups of incremental increase in baseline HbA1c allowed an evaluation of the clinical relevance of the observed effects, as the three groups represents three incremental level of HbA1c1 in T2D: Group A: HbA1c<53 mmol/mol: well regulated, no additional interventions needed; Group B: 64 mmol/mol>HbA1c≥53 mmol/mol; slightly dysregulated—increase nonpharmacoligal efforts and with extra control; and Group C: HbA1c≥64 mmol/mol: moderately dysregulated adjustment of medication to be considered.

### Cohen Effect Size

Cohen effect size was used as supplementary analyses to describe the size of the clinical effect.

The Cohen effect size was calculated as the difference in mean values for pre- and post treatment measurement for Active and Control groups divided by the pooled standard deviation. In relation to clinical significance, it has been suggested that a Cohen effect size<0.2 represents a minor clinical effect; 0.2-0.4: small effect; 0.4-0.7: moderate effect and ≥0.7: large effect.

### Intervention Procedure

This has been described previously (Faber et al. 2021). All participants received standard care according to national guidelines for the treatment of T2D, which included medical counselling by the study nurse, medical standardization, education in lifestyle adjustments.

Control group: The individuals in this group were informed that their PPS was elevated, which could reflect a physiological strain on the body and thus on their disease.

Active group: All subjects were receiving a non-pharmacological self-care stress management intervention program (UllCare®) and were instructed by a professional instructor.

This non-pharmacological intervention program has been described previously in detail (Ballegaard et al., 2014; Bergmann et al., 2014), and has the following basic elements: (i) a self-care part; (ii) a professional instruction in the PPS measurement, cognitive reflection in relation to the PPS measure and how to conduct sensory nerve stimulation, and (iii) continuous ongoing professional surveillance of the PPS measure allowing the possibility for pro-active professional contact if PPS measurements are missing or deviating.

The Self-care part consists of two daily and mandatory efforts in the morning and evening: (i) to perform PPS measurement, (ii) followed by sensory nerve stimulation as a mandatory stress-reducing procedure, (iii) reflection on both the PPS level and on general feeling of need for additional stress handling on a voluntary basis.

All subjects received a personal PPS measurement instrument, together with an instruction manual and were initially instructed during a two-hour group session with 5-10 participants in each group. This included education in performing PPS measurement, sensory nerve stimulation, and the theoretical background for the measurement as well as the intervention.

The participants were offered two individual appointments after 1 and 3 months, and 5 phone contacts after 1, 3, 5, 6 and 10 weeks, and similarly a two-hour group session every other month.

The participants were instructed to report their PPS measurements each day on their personal login on the website www.songdance.org. On the website each participant was able to track results and changes in PPS during the intervention period.

### Statistics

In order to assess the homeostatic effect, multivariable regression was applied with change in the outcome from baseline to after treatment as the dependent variable and the baseline outcome, treatment and the interaction between the two as independent variables. The homeostatic effect is presented as the difference in slopes between treated and untreated patients (or between responders and non-responders). In subsequent analyses the baseline outcomes were categorized in three categories and two (or three) way ANOVA was performed on the same dependent variables. Model validation was done by inspecting plots of standardized residuals against the fitted values and QQ-plots of the standardized residuals.

### Results

Patient characteristics at baseline are shown in Table 2.

TABLE 2
Baseline characteristics of randomized participants. No significant
differences between active and control group. BP: Blood Pressure.

| | | Active | | Control | |
|---|---|---|---|---|---|
| Number of persons | | 71 | | 73 | |
| Age (years); mean (range) | | 64.3 | (38-75) | 65.6 | (45-77) |
| Sex: male/female, (number) | | 46/25 | | 45/28 | |
| Diabetes duration (years) | | 10.6 | (1-25) | 9.9 | (1-24) |
| Primary diabetes control unit: | | 77% | | 78% | |
| General Practitioner (%) | | | | | |
| Medication (number (%)): | | | | | |
| 1. | Metformin | 54 | (76) | 56 | (77) |
| 2. | Insulin | 13 | (18) | 11 | (15) |
| 3. | GLP-1 agonist | 12 | (17) | 14 | (19) |
| 4. | SGLT-2 inhibitor | 8 | (11) | 11 | (15) |
| 5. | DPP-4 inhibitor | 10 | (14) | 20 | (27) |
| 6. | Sulfonylureas | 5 | (7) | 5 | (7) |
| 7. | Statins | 53 | (75) | 52 | (71) |
| 8. | ACE/ARB | 42 | (59) | 41 | (56) |
| 9. | Diuretics | 26 | (37) | 18 | (25) |
| 10. | Calciumchannel blockers | 24 | (34) | 16 | (22) |
| 11. | ASA | 22 | (31) | 20 | (27) |
| Medical history (number (%) ): | | | | | |
| 1. | Peripheral arterial disease | 3 | (4.2) | 1 | (1.4) |
| 2. | Previous myocardial infarction | 4 | (5.6) | 2 | (2.7) |
| 3. | Previous CABG or PCI | 6 | (8.5) | 2 | (2.8) |
| 4. | Previous stroke | 2 | (2.8) | 1 | (1.4) |
| 5. | Previous treated depression | 7 | (9.9) | 9 | (12) |
| 6. | Asthma | 11 | (15.5) | 3 | (4.2) |
| 7. | Previous cancer | 8 | (11.3) | 5 | (6.8) |
| 8. | Symptomatic neuropathy | 10 | (14) | 14 | (19) |
| | (number (%)) | | | | |

| | | | | |
|---|---|---|---|---|
| Pressure Pain Sensitivity (PPS) | | | | |
| (arbitrary units; mean (SD): | | | | |
| Sternum | 76.9 | (13.3) | 76.8 | (13.6) |
| Biochemistry (mean (SD)): | | | | |
| Hemoglobin A1C (HbA1C) | 53.7 | (8.6) | 53.6 | (10.5) |
| (mmol/mmol) | | | | |
| Creatinine (μmol/l) | 75.7 | (17.1) | 77.6 | (24.3) |
| Cholesterol (mmol/l) | 4.09 | (0.99) | 4.09 | (0.96) |
| LDL-cholesterol (mmol/l) | 2.39 | (3.52) | 2.05 | (0.77) |
| HDL-cholesterol (mmol/l) | 1.22 | (0.39) | 1.28 | (0.33) |
| Triglyceride (mmol/l) | 2.16 | (1.10) | 1.83 | (0.92) |
| Physiology (mean (SD)): | | | | |
| Systolic BP (mmHg) | 135 | (15) | 135 | (17) |
| Diastolic BP (mmHg) | 79 | (8) | 79 | (7) |
| Heart rate (beats/min) | 70 | (12) | 68 | (9) |
| BMI; (weight(kg)/height (m)² | 29.2 | (4.7) | 28.0 | (4.5) |

When the effect from the Active treatment was compared to that of the Control treatment for all 112 patients, mean HbA1c (mmol/mol) in the Active group (N=52) was reduced from 53.8 (SD: 8.4) to 50.5 (SD: 7.0) (intra-group P=0.001), compared to 53.8 (SD: 10.8) to 53.4 (10.9) in the Control group (N=60) (between-group P=0.04) (Cohen effect size: 0.31).

Based on the pre-study stratification of patients with respect to HbA1c three groups were defined: Group A: HbA1c<52 mmol/mol; B: HbA1c≥53 mmol/mol and <64 mmol/mol; and C: HbA1c≥64 mmol/mol.

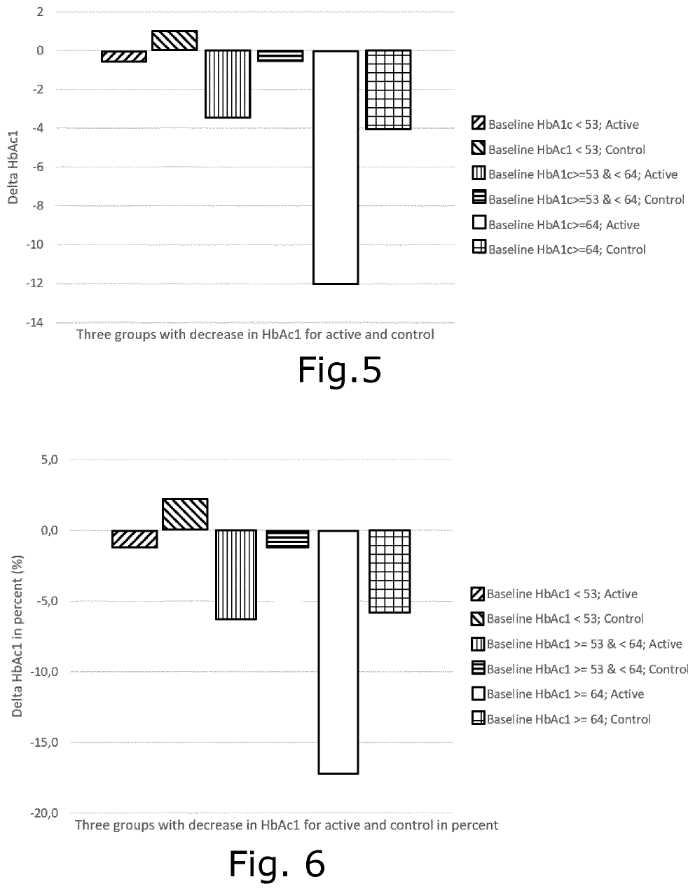

Table 3 and FIGS. 5 and 6 show the data and results when Active and Control groups were compared for each of these three groups.

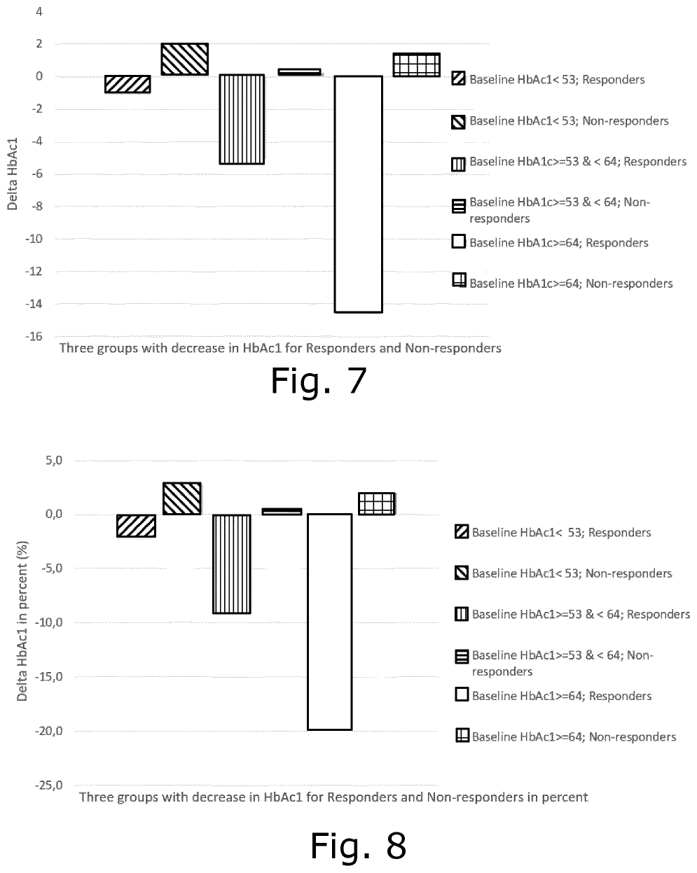

Table 3: Homeostasis effect on HbA1c measured at baseline and after three months of intervention, Active group compared to Control group, and for three groups of patients based on baseline HbA1c; Group A) all patients with HbA1c<53 mmol/mol; Group B) all patients with 53 mmol/mol≤HbA1c<64 mmol/mol; Group C) all patients with HbA1c≥64 mmol/mol). For explanation of Cohen effect size (see Text). The data of this table are shown in FIG. 7 as well.

TABLE 3A
Group A Baseline HbA1c < 52 mmol/mol

| | | Baseline | Post treatment | Between group | |
|---|---|---|---|---|---|
| | | HbA1c: | HbA1c: score | P-value | Cohen |
| | | Mean (S.D) | Mean (S.D) | (post- | effect |
| | n | mmol/mol | mmol/mol | treatment) | size |
| Active | 29 | 48.1 | 47.5 | N.S | 0.3 |
| | | (2.7) | (5.0) | | |
| Control | 34 | 46.7 | 47.7 | | |
| | | (3.6) | (6.4) | | |

TABLE 3B
Patients with 64 mmol/mol > HbA1c ≥ 53 mmol/mol.

| | | Baseline | Post treatment | Between group | |
|---|---|---|---|---|---|
| | | HbA1c: | HbA1c: score | P- value | Cohen |
| | | Mean (S.D) | Mean (S.D) | (post- | effect |
| | n | mmol/mol | mmol/mol | treatment) | size |
| Active | 15 | 56.1 | 52.6 | N.S | 0.6 |
| | | (2.9) | (8.1) | | |
| Control | 15 | 55.7 | 55.1 | | |
| | | (2.6) | (5.4) | | |

TABLE 3C
Patients with HbA1c ≥ 64 mmol/mol.

| | | Baseline | Post treatment | Between group | |
|---|---|---|---|---|---|
| | | HbA1c: | HbA1c: score | P-value | Cohen |
| | | Mean (S.D) | Mean (S.D) | (post- | effect |
| | n | mmol/mol | mmol/mol | treatment) | size |
| Active | 8 | 70.0 | 58.0 | 0.03 | 1.1 |
| | | (5.1) | (4.1) | | |
| Control | 11 | 73.1 | 69.0 | | |
| | | (7.2) | (12.2) | | |

Regarding the clinical effect size as measured by Cohens factor, this was increasing with increasing baseline HbA1c, being 1.1 in group C.

Based on a pre-study defined minimal clinically relevant reduction of PPS (i.e. 15 arbitrary unit), the study was divided in a group of Responders (who obtained this reduction in PPS) (N=52) and a group of Non-responders (N=60) who did not obtain this reduction.

When all Responders were compared to all Non-responders, the group of Responders had a mean reduction in HbA1c (mmol/mol) from 54.9 (SD: 11.0) to 49.9 (SD: 7.4), compared to a mean increase from 52.8/(SD: 8.5) to 53.9 (SD: 12.5) in the Control Group (Between-group difference P<0.0001) (Cohen effect size: 0.62).

Table 4 and FIGS. 7 and 8 show the changes of HbA1c during the study period for Responders and Non-responders, and for the three levels of incremental increase in baseline HbA1c.

Table 4: Homeostasis effect on HbA1c measured at baseline and after three months of intervention, Group of Responders compared to group of Non-responders, and for three groups of patients based on baseline HbA1c; Group A) all patients with HbA1c<53 mmol/mol; Group B) all patients with 53 mmol/mol≤HbA1c<64 mmol/mol; Group C) all patients with HbA1c≥64 mmol/mol). For explanation of Cohen effect size (see text). The data of this table are shown in FIG. 7 as well.

TABLE 4A
Group A Baseline HbA1c < 53 mmol/mol

| | | Baseline | Post treatment | Between group | |
|---|---|---|---|---|---|
| | | HbA1c: | HbA1c: | P-value | Cohen |
| | | Mean (S.D) | Mean (S.D) | (post- | effect |
| | n | mmol/mol | mmol/mol | treatment) | size |
| Responder | 28 | 47.4 | 46.3 | 0.1 | 0.6 |
| | | (3.3) | (4.2) | | |
| Non- | 35 | 47.3 | 48.6 | | |
| responder | | (3.3) | (6.5) | | |

TABLE 4B
Patients with 64 mmol/mol > HbA1c ≥ 53 mmol/mol.

| | | Baseline | Post treatment | Between group | |
|---|---|---|---|---|---|
| | | HbA1c: | HbA1c: score | P-value | Cohen |
| | | Mean (S.D) | Mean (S.D) | (post- | effect |
| | n | mmol/mol | mmol/mol | treatment) | size |
| Responder | 13 | 55.6 | 50.5 | 0.016 | 3.0 |
| | | (2.2) | (5.4) | | |
| Non- | 17 | 56.2 | 56.4 | | |
| responder | | (3.1) | (6.9) | | |

TABLE 4C
Patients with HbA1c ≥ 64 mmol/mol.

| | | Baseline | Post treatment | Between group | |
|---|---|---|---|---|---|
| | | HbA1c: | HbA1c: score | P-value | Cohen |
| | | Mean (S.D) | Mean (S.D) | (post- | effect |
| | n | mmol/mol | mmol/mol | treatment) | size |
| Responder | 11 | 73.2 | 58.6 | 0.01 | 14.6 |
| | | (7.6) | (8.6) | | |
| Non- | 8 | 69.9 | 71.4 | | |
| responder | | (4.2) | (10.2) | | |

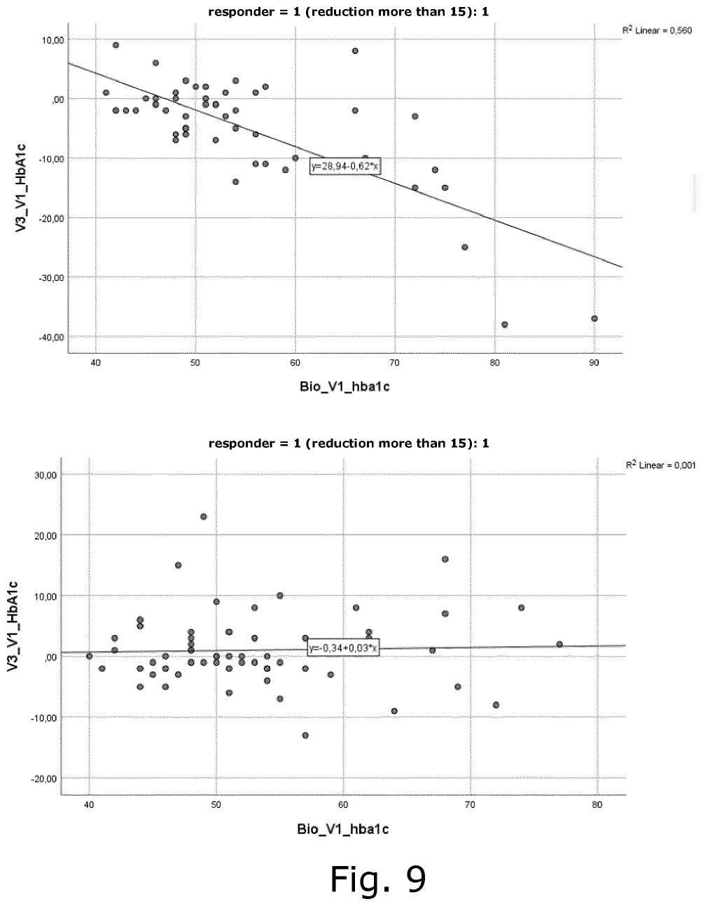

With respect to examine the homeostatic effect from correlation analysis between baseline HbA1c versus changes in HbA1c during the intervention period, responders obtained a homeostatic response measured as the coefficient of correlation (r)=0.75 (N=52) (intragroup P<0.0001), compared to r=−0.04 for non-responders (N=60) (between group P<0.0001) (FIG. 9).

When the homeostatic effect was measured from regression analysis and by comparing Responders versus Non-responders, the mean homeostatic effect of 0.57 mmol/mol for every 1 mmol/mol increase in baseline HbA1c (95% confidence interval: 0.31-0.83 mmol/mol (P<0.001)

Results in brief: When compared to non-responders, the responders obtained a mean homeostatic effect of 0.57 mmol/mol for every 1 mmol/mol increase in baseline HbA1c (95% confidence interval: 0.31-0.83 mmol/mol (P<0.001). For responders with a pre-defined minimum elevation of HbA1c (i.e., HbA1c≥64 mmol/mol), the reduction of HbA1c was 22% compared to non-responders (P<0.01).

### Conclusions

We showed that non-pharmacological normalization of elevated Pressure Pain Sensitivity (PPS) at the chest bone in patients with Type 2 Diabetes (T2D) is associated with renewed glucose metabolic homeostasis expressed by normalization of elevated HbA1c measures in blood plasma. The proposed approach to the reversal of glucose homeostasis is a new supplementary and clinically useful key to the understanding, prevention and treatment of T2D.

The primary outcome measure chosen (HbA1c) was not influenced by participant or researcher bias; secondly, the hypothesis that reducing an elevated PPS by the present intervention has a homeostatic effect was predefined and previously tested positively in healthy subjects (Ballegaard et al 2014). Thirdly, the Control group was not a genuine control group since they did not receive placebo treatment, but rather were informed that the measured elevated PPS represented a signal of as potential unhealthy state. This could have inspired for therapeutic measures by the persons of the Control group, which may explain, that approximately 25% of the controls obtained a clinical relevant reduction of the elevated PPS. Usually, this is regarded as a potential bias, and it may increase the risk of a type 2 error. This serves to underscore the relevance of our findings of a progressive reduction in HbA1c with increasing basal HbA1c. The responders had a pronounced effect in terms of enhanced homeostatic effect independently of being randomized to Active or Control group. In terms of likelihood of becoming a responder, the likelihood was five times higher for the Active group, compared to the Control group. The difference is consistent with the conceptual understanding that the reduction of HbA1c primarily is associated with a reduction of PPS and thus with ANSD.

Elevated PPS is quite prevalent in T2D, approximately 60-70% (Faber et al 2021). The treatment with repeated sensory nerve stimulation guided by home based PPS measurements and followed by continuous professional surveillance seems to have clinically relevant effects reducing blood glucose levels, especially among those people with HbA1c above 64 mmol/mol. In these subjects which may be regarded as moderately dysregulated, a reduction in HbA1c of approximately 20% can be expected, if PPS can be reduced, i.e. the person becoming a responder. Therefore, measuring of PPS on a regular basis in diabetic clinics, for instance yearly, and taking action when elevated, identifying potential responders and providing guidance, may represent a new way of viewing and treating the T2D disease. Regular PPS measuring could complement the existing program for treating T2D. Concerning the actions regarding prevention of diabetic complications, which has great attention during the follow-up of the patients, it might be a strengthen to the contemporary treatments also to measure PPS, elevated PPS is associated with the presence of cardiovascular autonomic neuropathy (CAN) (Faber et al), which is known to be associated with increasing morbidity and mortality in T2D (Spallone 2019). Also, a reduction of PPS, as shown in randomized clinical trials (RCTs), seems to improve important health risk factors in T2D patients, such a depression (Bergmann et at 2014, Ballegaard et al 2016), heart rate, blood pressure, work of the heart, serum cholesterol (Ballegaard et al 2014), and blood pressure response to a tilt table test (Ballegaard et al 2015).

In healthy subjects reduction of an elevated PPS resulted in a reduction in HbA1c (Ballegaard et al 2014). As such, tracing and treating of an elevated PPS in prediabetes, as for example seen in obesity, might also turn out to have positive effect on the risk of developing T2D. Clearly, this calls for additional research.

### Example 3

### Intervention Effect on IHD-Related Depression

Depression in patients suffering from IHD is known to be associated with increased mortality rates, and ANSD may link the two conditions. The inventor has demonstrated that PPS at the sternum increases with increased depression in IHD, and that reduction of elevated PPS measurements reflects:
lower depression scores, ANSD, and mortality rates in IHD, and
regulatory functions of the autonomic nervous system with respect to IHD risk factors including heart rate, blood pressure, serum cholesterol, and glycated haemoglobin.

However, beta-adrenoreceptor blockade (BB) is found to inhibit the anti-depressive effect associated with reduced PPS measures.

It was therefore hypothesized that lowering of elevated PPS measures will be associated with enhanced autonomic regulation of depression, measured as an association between baseline depression scores and subsequent lowering of depression scores.

The enhanced autonomic regulation is hypothetically expressed as a homeostatic normalization of depression score, with the understanding that higher levels of baseline depression score are associated with larger reduction of depression score as PPS is reduced. This is indicated by intensifying of the normalization mechanisms influenced by the autonomic nervous system when baseline depression score is at higher levels.

In a randomized clinical trial of IHD patients, which included 165 patients with elevated PPS, 20% exhibited elevated Major Depression Inventory (MDI) scores. Subjects with previously defined minimum reduction of at least 15 PPS units (PPS reverters) were compared with subjects without this effect (PPS non-reverters), as well as non-users versus users of BB. We performed linear regressions of depression score changes as dependent variable and baseline depression scores as independent variable.

It was found that for (N=72) compared to (N=93), the mean anti-depressive effect difference between the two groups increased by an average rate of 0.31 MDI units (95% CI 0.10-0.52, P=0.004) for every one unit of increase in baseline depression score. Comparing BB non-users (N=97) with BB users (N=67), the average rate of increase of the difference was 0.37 units (95% CI 0.18-0.56, P=0.0002). In combination, rate of increase of the difference of 0.70 (95% CI 0.43-0.97, P<0.00001). For non-BB-users with elevated MDI scores at baseline (>15) and response to treatment by at least 15 PPS units, Cohen's effect size was 1.5.

It was hence concluded that in patients with IHD, depression is associated with disruption of specific regulatory functions of the autonomic nervous system. This finding implies that lowering of elevated PPS measures indicates enhanced autonomic lowering of depression scores.

### LIST OF REFERENCES

1) Abdelhamid, A. S., Brown, T. J., Brainard, J. S., Biswas, P., Thorpe, G. C., Moore, H. J., et al. (2018). Omega-3 fatty acids for the primary and secondary prevention of cardiovascular disease. Cochrane Database Syst Rev 7, CD003177. doi: 10.1002/14651858.CD003177.pub3.
2) Anderson, L., Oldridge, N., Thompson, D. R., Zwisler, A.-D., Rees, K., Martin, N., et al. (2016). Exercise-Based Cardiac Rehabilitation for Coronary Heart Disease: Cochrane Systematic Review and Meta-Analysis. J Am Coil Cardiol 67, 1-12. doi:10.1016/j.jacc.2015.10.044.
3) Ballegaard, S., Bergmann, N., Karpatschof, B., Kristiansen, J., Gyntelberg, F., Arendt-Nielsen, L., et al. (2015). Association between pressure pain sensitivity and autonomic function as assessed by a tilt table test. Scand. J. Clin. Lab. Invest. 75, 345-354. doi: 10.3109/00365513.2015.1028095.
4) Ballegaard, S., Bergmann, N., Karpatschof, B., Kristiansen, J., Gyntelberg, F., Arendt-Nielsen, L., et al. (2016). Association between Depression, Pressure Pain Sensitivity, Stress and Autonomous Nervous System Function in Stable Ischemic Heart Disease: Impact of Beta-Adrenergic Receptor Blockade. Journal of Behavioral and Brain Science 6, 317-328. doi:10.4236/jbbs.2016.68031.
5) Ballegaard, S., Borg, E., Karpatschof, B., Nyboe, J., and Johannessen, A. (2004). Long-term effects of integrated rehabilitation in patients with advanced angina pectoris: a nonrandomized comparative study. J Altern Complement Med 10, 777-783. doi:10.1089/acm.2004.10.777.
6) Ballegaard, S., Petersen, P. B., Harboe, G. S., Karpatschof, B., Gyntelberg, F., and Faber, J. (2014). The association between changes in pressure pain sensitivity and changes in cardiovascular physiological factors associated with persistent stress. Scandinavian Journal of Clinical and Laboratory Investigation 74, 116-125. doi: 10.3109/00365513.2013.862847.
7) Bech P, “Clinical Psychometrics”; Oxford: Wiley Blackwell; 2012
8) Bech P et al. (2015), BMC Psychiatry 15:190 (doi: 10.1186/s12888-015-0529-3).
9) Bergmann, N., Ballegaard, S., Bech, P., Hjalmarson, A., Krogh, J., Gyntelberg, F., et al. (2014). The effect of daily self-measurement Of pressure pain sensitivity followed by acupressure on depression and quality of life versus treatment as usual in ischemic heart disease: a randomized clinical trial. PLoS ONE 9, e97553. doi:10.1371/journal.pone.0097553.
10) Bhati, P., Alam, R., Moiz, J. A., and Hussain, M. E. (2019). Subclinical inflammation and endothelial dysfunction are linked to cardiac autonomic neuropathy in type 2 diabetes. J Diabetes Metab Disord 18, 419-428. doi:10.1007/s40200-019-00435-w.
11) Carnevali, L., Koenig, J., Sgoifo, A., and Ottaviani, C. (2018). Autonomic and Brain Morphological Predictors of Stress Resilience. Front Neurosci 12, 228. doi: 10.3389/fnins.2018.00228.
12) Chalaye, P., Goffaux, P., Bourgault, P., Lafrenaye, S., Devroede, G., Watier, A., et al. (2012). Comparing pain modulation and autonomic responses in fibromyalgia and irritable bowel syndrome patients. Clin J Pain 28, 519-526. doi:10.1097/AJP.0b013e31823ae69e.
13) Cox, D. R., Spjøtvoll, E., Johansen, S., van Zwet, W. R., Bithell, J. F., Barndorff-Nielsen, O., et al. (1977). The Role of Significance Tests [with Discussion and Reply]. Scandinavian Journal of Statistics 4, 49-70.
14) Dong, S.-Y., Yan, S.-T., Wang, M.-L., Li, Z.-B., Fang, L.-Q., and Zeng, Q. (2018). Associations of body weight and weight change with cardiovascular events and mortality in patients with coronary heart disease. Atherosclerosis 274, 104-111. doi:10.1016/j.atherosclerosis.2018.05.007.
15) Faber, J., Eldrup, E., Selmer, C., Pichat, C., Hecquet, S. K., Watt, T., et al. (2021). Reduction of Pressure Pain Sensitivity as Novel Non-pharmacological Therapeutic Approach to Type 2 Diabetes: A Randomized Trial. Front. Neurosci. 15. doi: 10.3389/fnins.2021.613858.
16) Grippo, A. J., and Johnson, A. K. (2009). Stress, depression and cardiovascular dysregulation: a review of neurobiological mechanisms and the integration of research from preclinical disease models. Stress 12, 1-21. doi:10.1080/10253890802046281.
17) Güemes, A., and Georgiou, P. (2018). Review of the role of the nervous system in glucose homoeostasis and future Perspectives towards the management of diabetes. Bioelectron Med 4. doi:10.1186/s42234-018-0009-4.
18) Hooper, L., Martin, N., Jimoh, O. F., Kirk, C., Foster, E., and Abdelhamid, A. S. (2020). Reduction in saturated fat intake for cardiovascular disease. Cochrane Database Syst Rev 5, CD011737. doi:10.1002/14651858.CD011737.pub2.
19) Koenig, H. G. (2012). Religion, spirituality, and health: the research and clinical implications. ISRN Psychiatry 2012, 278730. doi:10.5402/2012/278730.
20) Marso, S. P., Daniels, G. H., Brown-Frandsen, K., Kristensen, P., Mann, J. F. E., Nauck, M. A., et al. (2016). Liraglutide and Cardiovascular Outcomes in Type 2 Diabetes. N Engl J Med 375, 311-322. doi:10.1056/NEJMoa1603827.
21) McEwen, B. S. (2016). In pursuit of resilience: stress, epigenetics, and brain plasticity. Ann N YAcad Sci 1373, 56-64. doi:10.1111/nyas.13020.
22) McEwen, B. S., Nasca, C., and Gray, J. D. (2016). Stress Effects on Neuronal Structure: Hippocampus, Amygdala, and Prefrontal Cortex. Neuropsychopharmacology 41, 3-23. doi: 10.1038/npp.2015.171.
23) Novak, P. (2011). Quantitative autonomic testing. J Vis Exp. doi:10.3791/2502. Prabhakaran, D., Chandrasekaran, A. M., Singh, K., Mohan, B., Chattopadhyay, K., Chadha, D. S., et al. (2020). Yoga-Based Cardiac Rehabilitation After Acute Myocardial Infarction: A Randomized Trial. J Am Coil Cardiol 75, 1551-1561. doi:10.1016/j.jacc.2020.01.050.
24) Quinones, A., Lobach, I., Maduro, G. A., Smilowitz, N. R., and Reynolds, H. R. (2015). Diabetes and ischemic heart disease death in people age 25-54: a multiple-cause-of-death analysis based on over 400 000 deaths from 1990 to 2008 in New York City. Clin Cardiol 38, 114-120. doi: 10.1002/clc.22367.
25) Rees, K., Takeda, A., Martin, N., Ellis, L., Wijesekara, D., Vepa, A., et al. (2019). Mediterranean-style diet for the primary and secondary prevention of cardiovascular disease. Cochrane Database Syst Rev 3, CD009825. doi: 10.1002/14651858.CD009825.pub3.
26) Richards, S. H., Anderson, L., Jenkinson, C. E., Whalley, B., Rees, K., Davies, P., et al. (2018). Psychological interventions for coronary heart disease: Cochrane systematic review and metaanalysis. Eur J Prev Cardiol 25, 247-259. doi:10.1177/2047487317739978.
27) Richner, M., Pallesen, L. T., Ulrichsen, M., Poulsen, E. T., Holm, T. H., Login, H., et al. (2019). Sortilin gates neurotensin and BDNF signaling to control peripheral neuropathic pain. Sci Adv 5, eaav9946. doi:10.1126/sciadv.aav9946.
28) Rosmond, R. (2005). Role of stress in the pathogenesis of the metabolic syndrome. Psychoneuroendocrinology 30, 1-10. doi:10.1016/j.psyneuen.2004.05.007.
29) Schneiderman, N., Saab, P. G., Catellier, D. J., Powell, L. H., DeBusk, R. F., Williams, R. B., et al. (2004). Psychosocial treatment within sex by ethnicity subgroups in the Enhancing Recovery in Coronary Heart Disease clinical trial. Psychosom Med 66, 475-483. doi:10.1097/01.psy.0000133217.96180.e8.
30) Sidney, S., Sorel, M. E., Quesenberry, C. P., Jaffe, M. G., Solomon, M. D., Nguyen-Huynh, M. N., et al. (2018). Comparative Trends in Heart Disease, Stroke, and All-Cause Mortality in the United States and a Large Integrated Healthcare Delivery System. Am. J. Med. 131, 829-836.e1. doi:10.1016/j.amjmed.2018.02.014.
31) Spallone, V. (2019). Update on the Impact, Diagnosis and Management of Cardiovascular Autonomic Neuropathy in Diabetes: What Is Defined, What Is New, and What Is Unmet. Diabetes Metab J 43, 3-30. doi: 10.4093/dmj.2018.0259.
32) Staufenbiel, S. M., Penninx, B. W. J. H., Spijker, A. T., Elzinga, B. M., and van Rossum, E. F. C. (2013). Hair cortisol, stress Exposure, and mental health in humans: a systematic review. Psychoneuroendocrinology 38, 1220-1235. doi:10.1016/j.psyneuen.2012.11.015.
33) Steptoe, A., and Kivimäki, M. (2012). Stress and cardiovascular disease. Nat Rev Cardiol 9, 360-370. doi: 10.1038/nrcardio.2012.45.
34) Tesfaye, S., Boulton, A. J. M., Dyck, P. J., Freeman, R., Horowitz, M., Kempler, P., et al. (2010). Diabetic neuropathies: update on Definitions, diagnostic criteria, estimation of severity, and treatments. Diabetes Care 33, 2285-2293. doi:10.2337/dc10-1303.
35) Thayer, J. F., and Lane, R. D. (2007). The role of vagal function in the risk for cardiovascular disease and mortality. Biol Psychol 74, 224-242. doi:10.1016/j.biopsycho.2005.11.013.
36) Uvnäs-Moberg, K., Handlin, L., and Petersson, M. (2014). Self-soothing behaviors with particular reference to oxytocin release induced by non-Noxious sensory stimulation. Front Psychol 5, 1529. doi:10.3389/fpsyg.2014.01529.
37) Wentworth, B. A., Stein, M. B., Redwine, L. S., Xue, Y., Taub, P. R., Clopton, P., et al. (2013). Posttraumatic stress disorder: a fast track to premature Cardiovascular disease? Cardiol Rev 21, 16-22. doi:10.1097/CRD.0b013e318265343b.
38) Tobaldini G. et al. (2020). Pain and stress: functional evidence that supra-spinal mechanisms involved in pain-induced analgesia mediate stress-induced analgesia. Behav Pharmacol 31 (2&3):159-167. doi: 10.1097/FBP.0000000000000529.
39) Askari K, Oryan S, Eidi A, Zaringhalam J, Haghparast A. (2021). Modulatory role of the orexin system in stress-induced analgesia: Involvement of the ventral tegmental area. Eur J Pain 25(10):2266-2277. doi: 10.1002/ejp.1840. Epub 2021 Jul. 27.
40) Thomson J S, Deakin A G, Cossins A R, Spencer J W, Young I S, Sneddon L U, C. Bernard (1878-1879). Acute and chronic stress prevents responses to pain in zebrafish: evidence for stress-induced analgesia. Lecons sur les phenomenes de la vie communs aux animaux et aux vegetaux. 2 vols. Paris: Bailliere, 1878-1879.Google Scholar
41) W. B. Cannon: In. A. Pettit (ed.). A Charles Richet: ses amis, ses collegues, ses eleves, p. 91. Paris: Editions Medicales, 1926. (scholar.qooqle.com/scholar lookup?&author=WB.%20Cannon&paqes=91&publication year=1926).
42) H. Selye (1971). Hormones and Resistance. Heidelberg: Springer-Verlag, 1971. (scholar.qooqle.com/scholar lookup?title=Hormones %20and %20Resistance&author=H.%20Selve&publication_year=1971).
43) Diabetes Prevention Program (DPP) Research Group (2002). The Diabetes Prevention Program (DPP): description of lifestyle intervention. Diabetes Care 25(12):2165-71. doi: 10.2337/diacare.25.12.2165.PMID: 12453955.
44) Sevick M A, Woolf K, Mattoo A, Katz S D, Li H, St-Jules D E, Jagannathan R, Hu L, Pompeii M L, Ganguzza L, Li Z, Sierra A, Williams S K, Goldfarb D S (2018). The Healthy Hearts and Kidneys (HHK) study: Design of a 2×2 RCT of technology-supported self-monitoring and social cognitive theory-based counseling to engage overweight people with diabetes and chronic kidney disease in multiple lifestyle changes. Contemp Clin Trials 64:265-273. doi: 10.1016/j.cct.2017.08.020. Epub 2017 Sep. 1.
45) Adeghate E, Lotfy M, D'Souza C, Alseiari S M, Alsaadi A A, Qahtan S A (2020). Hypocretin/orexin modulates body weight and the metabolism of glucose and insulin. Diabetes Metab Res Rev. 2020 36(3):e3229. doi: 10.1002/dmrr.3229. Epub 2020 Jan. 16.
46) Tsuneki H, Wada T, Sasaoka T (2012). Role of orexin in the central regulation of glucose and energy homeostasis. Endocr J. 59(5):365-74. doi: 10.1507/endocrj.ej12-0030. Epub 2012 Jan. 28.

1. A method for determining whether
1) a human subject has a lowered expected survival rate, comprising establishing, via at least one measurement of exposure to a quantifiable pain-inducing influence, the subject's pain threshold at one or more sympathetic tone dependent locations on or in the body, and determining that the human subject has a lowered expected survival rate if the subject's pain threshold is reduced compared to either a predetermined standard value or compared the subject's own pain threshold at sympathetic tone independent locations on or in the body, or
2) a human subject suffers or from or is at increased risk of contracting type 2 diabetes mellitus (T2D), which can be treated via non-pharmacologic intervention, comprising establishing, via at least one measurement of exposure to a quantifiable pain-inducing influence, the subject's pain threshold at one or more sympathetic tone dependent locations on or in the body, and determining that the T2D can be treated by non-pharmacological intervention, if the subject's pain threshold is reduced compared to either a predetermined standard value or compared the subject's own pain threshold at sympathetic tone independent locations on or in the body.

2. The method according to claim 1, wherein reduction in pain threshold at the sympathetic tone dependent location is reflected in a corresponding reduction in the measurement value of the exposure to the quantifiable pain-inducing influence.

3. The method according to claim 1, option 2, wherein the human subject in addition to the lowered pain threshold also exhibits an elevated HbA1c blood concentration.

4. The method according to claim 1, wherein the quantifiable pain-inducing influence is selected from heat, localized physical pressure, and electricity, preferably localized physical pressure.

5. The method according to claim 4, wherein the measurement of exposure to the quantifiable pain-inducing influence comprises at least one measurement of pressure point sensitivity (PPS).

6. The method according to claim 1, option 1, wherein the human subject a priori has a lowered expected survival rate due to an illness.

7. The method according to claim 6, wherein the illness is diabetes mellitus (type I or type II), cancer, ischemic heart disease (IHD), or IHD-related depression, preferably IHD or IHD-related depression.

8. The method according to claim 1, wherein the one or more sympathetic tone dependent locations is or comprises the periosteum of the anterior part of the sternum.

9. A method of
1) increasing the life expectancy of a human subject, the method comprising determining, according to the method of claim 1, that the subject has a lowered expected survival rate, and subsequently subjecting the subject to a treatment, which increases the subject's pain threshold a sympathetic tone dependent locations, or
2) treating a patient suffering from diabetes mellitus type 2, the method comprising determining, according to the method of claim 1, that the subject has or is at increased risk of contracting T2D that can be treated or prevented via non-pharmacological intervention, and subsequently subjecting the subject to a treatment, which increases the subject's pain threshold a sympathetic tone dependent locations whereby a reduction in the HbA1c blood concentration is attained, or
3) treating a patient suffering from IHD-related depression, the method comprising determining, according to the method of claim 1, that the subject suffering from the IHD-related depression has a lowered expected survival rate, and subsequently subjecting the subject to a treatment, which increases the subject's pain threshold a sympathetic tone dependent locations.

10. The method according to claim 9, wherein the treatment's efficacy is verified by repeatedly measuring the subject's pain threshold and ensuring that the pain threshold is increased as evidenced by an increase over time of measurement values of the quantifiable pain-inducing influence.

11. The method according to claim 9, wherein the human subject is as defined in claim 6 or 7 or a subject who does not a priori have a lowered expected survival rate.

12. The method according to claim 9, wherein the treatment comprises repeated, such as daily, autonomic reflex activation and/or personal empowerment.

13. The method according to claim 12, wherein the repeated autonomic reflex activation comprises peripheral non-noxious sensory nerve stimulations at a sympathetic tone dependent point, preferably involving the anterior sternal periosteum, or at any other locations within the spinal segmental innervation area of the relevant measurement site or at other locations outside these areas with a neurological connection to the measurement site, for repeated autonomic reflex elevation of the noxious withdrawal reflex threshold, and wherein the personal empowerment comprises free-choice physical and mental exercises.

14. The method according to claim 8, which comprises repeated PPS measurements and optionally ongoing PPS measurements evaluation address missing or deviating PPS measurements.

15. A method for identifying a therapeutic treatment, which has a high likelihood of reducing or curing or preventing ANSD or T2D or IHD-related depression, the method comprising via at least two measurements of exposure to a quantifiable pain-inducing influence, a determination of at least one subject's pain threshold at one or more sympathetic tone dependent locations on or in the body, subsequently identifying the treatment as one that has the high likelihood of reducing or curing or preventing ANSD or T2D or IHD-related depression if the at least one subject exhibits a significant increase in the pain threshold at the one or more sympathetic tone dependent locations when receiving the treatment.

16. The method according to claim 15, wherein the at least one subject at the onset exhibits a reduced pain threshold at the one or more sympathetic tone dependent locations.

17. The method according to claim 15, which comprises repeatedly, such as daily determining the at least one subject's pain threshold.

18. The method according to claim 15, wherein the at least one measurement is carried out as in claim 2.

19. The method according to claim 15, wherein the at least one subject is a plurality of subjects.

20. A method for evaluating efficacy of a treatment or prophylactic treatment of ANSD or T2D or IHD-related depression, the method comprising via at least two measurements of exposure to a quantifiable pain-inducing influence, a determination of at least one subject's pain threshold at one or more sympathetic tone dependent locations on or in the body, wherein said at least one subject is undergoing a treatment or prophylactic treatment of ANSD or T2D or IHD-related depression, and verifying that the treatment reduces or cures or prevents ANSD or T2D or IHD-related depression if the at least one subject exhibits a significant increase in the pain threshold at the one or more sympathetic tone dependent locations.

21. The method according to claim 20, wherein the at least one subject at the onset exhibits a reduced pain threshold at the one or more sympathetic tone dependent locations.

22. The method according to claim 20, which comprises repeatedly, such as daily determining the at least one subject's pain threshold.

23. The method according to claim 20, wherein the at least one measurement is carried out as in claim 2.
